(19) United States
(12) Patent Application Publication
(10) Pub. No.: US 2009/0247549 A1
(43) Pub. Date: Oct. 1, 2009

(54) Methods of using saha and bortezomib for treating cancer

(76) Inventors: Stanley R. Frankel, Yardley, PA (US); Paul J. Deutsch, Princeton, NJ (US); Sophia Randolph, Burien, WA (US); Bernard Fine, Menlo Park, CA (US)

Correspondence Address: MERCK AND CO., INC, P O BOX 2000, RAHWAY, NJ, 07065-0907, US

(21) Appl. No.: 12/154,087

(22) Filed: May 20, 2008

Related U.S. Application Data
(63) Continuation of application No. 11/592,528, filed on Nov. 3, 2006, now abandoned.
(60) Provisional application No. 60/733,951, filed on Nov. 4, 2005.

(51) Int. Cl. A61K 31/497; A61P 35/00

(52) U.S. Cl. 514/255.05

(57) ABSTRACT

The present invention relates to a method of treating cancer in a subject in need thereof, by administering to a subject in need thereof a first amount of a histone deacetylase (HDAC) inhibitor such as suberoylanilide hydroxamic acid (SAHA), or a pharmaceutically acceptable salt or hydrate thereof, and a second amount of one or more anti-cancer agents, including Bortezomib. The HDAC inhibitor and the anti-cancer agent may be administered to comprise therapeutically effective amounts. In various aspects, the effect of the HDAC inhibitor and the anti-cancer agent may be additive or synergistic.

### FIELD OF THE INVENTION

The present invention relates to a method of treating cancer by administering a histone deacetylase (HDAC) inhibitor such as suberoylanilide hydroxamic acid (SAHA) in combination with one or more anti-cancer agents, including Bortezomib. The combined amounts together can comprise a therapeutically effective amount.

### BACKGROUND OF THE INVENTION

Cancer is a disorder in which a population of cells has become, in varying degrees, unresponsive to the control mechanisms that normally govern proliferation and differentiation.

Therapeutic agents used in clinical cancer therapy can be categorized into several groups, including, alkylating agents, antibiotic agents, antimetabolic agents, biologic agents, hormonal agents, and plant-derived agents.

Cancer therapy is also being attempted by the induction of terminal differentiation of the neoplastic cells (M. B., Roberts, A. B., and Driscoll, J. S. (1985) in Cancer: Principles and Practice of Oncology, eds. Hellman, S., Rosenberg, S. A., and DeVita, V. T., Jr., Ed. 2, (J. B. Lippincott, Philadelphia), P. 49). In cell culture models, differentiation has been reported by exposure of cells to a variety of stimuli, including: cyclic AMP and retinoic acid (Breitman, T. R., Selonick, S. E., and Collins, S. J. (1980) Proc. Natl. Acad. Sci. USA 77: 2936-2940; Olsson, I. L. and Breitman, T. R. (1982) Cancer Res. 42: 3924-3927), aclarubicin and other anthracyclines (Schwartz, E. L. and Sartorelli, A. C. (1982) Cancer Res. 42: 2651-2655). There is abundant evidence that neoplastic transformation does not necessarily destroy the potential of cancer cells to differentiate (Sporn et al; Marks, P. A., Sheffery, M., and Rifkind, R. A. (1987) Cancer Res. 47: 659; Sachs, L. (1978) Nature (Lond.) 274: 535).

There are many examples of tumor cells which do not respond to the normal regulators of proliferation and appear to be blocked in the expression of their differentiation program, and yet can be induced to differentiate and cease replicating. A variety of agents can induce various transformed cell lines and primary human tumor explants to express more differentiated characteristics. Histone deacetylase inhibitors such as suberoylanilide hydroxamide acid (SAHA), belong to this class of agents that have the ability to induce tumor cell growth arrest, differentiation, and/or apoptosis (Richon, V. M., Webb, Y., Merger, R., et al. (1996) PNAS 93:5705-8). These compounds are targeted towards mechanisms inherent to the ability of a neoplastic cell to become malignant, as they do not appear to have toxicity in doses effective for inhibition of tumor growth in animals (Cohen, L. A., Amin, S., Marks, P. A., Rifkind, R. A., Desai, D., and Richon, V. M. (1999) Anticancer Research 19:4999-5006). There are several lines of evidence that histone acetylation and deacetylation are mechanisms by which transcriptional regulation in a cell is achieved (Grunstein, M. (1997) Nature 389:349-52). These effects are thought to occur through changes in the structure of chromatin by altering the affinity of histone proteins for coiled DNA in the nucleosome.

There are five types of histones that have been identified (designated H1, H2A, H2B, H3 and H4). Histones H2A, H2B, H3, and H4 are found in the nucleosomes and H1 is a linker located between nucleosomes. Each nucleosome contains two of each histone type within its core, except for H1, which is present singly in the outer portion of the nucleosome structure. It is believed that when the histone proteins are hypoacetylated, there is a greater affinity of the histone to the DNA phosphate backbone. This affinity causes DNA to be tightly bound to the histone and renders the DNA inaccessible to transcriptional regulatory elements and machinery. The regulation of acetylated states occurs through the balance of activity between two enzyme complexes, histone acetyl transferase (HAT) and histone deacetylase (HDAC). The hypoacetylated state is thought to inhibit transcription of associated DNA. This hypoacetylated state is catalyzed by large multiprotein complexes that include HDAC enzymes. In particular, HDACs have been shown to catalyze the removal of acetyl groups from the chromatin core histones.

Multiple myeloma, a B-cell malignancy of plasma cells, represents the second most common hematological malignancy. The annual incidence in the United States is about four per 100,000. Approximately 13,600 cases of multiple myeloma are diagnosed each year. Approximately 11,200 deaths per year are due to the disease, representing approximately 2% of all cancer deaths.

Multiple myeloma is characterized by the neoplastic proliferation of a single clone of plasma cells engaged in the production of a monoclonal immunoglobulin. Although multiple myeloma cells are initially responsive to radiotherapy and chemotherapy, durable complete responses are rare and virtually all patients who respond initially ultimately relapse. As the disease progresses, morbidity and eventual mortality are caused by lowering resistance to infection, significant skeletal destruction (with bone pain, pathological fractures and hypercalcemia), anemia, renal failure and hyperviscosity. To date, conventional treatment approaches have not resulted in long-term disease-free survival, which highlights the importance of developing new drug treatment for this incurable disease.

Besides the aim to increase the therapeutic efficacy, another purpose of combination treatment is the potential decrease of the doses of the individual components in the resulting combinations in order to decrease unwanted or harmful side effects caused by higher doses of the individual components. Thus, there is an urgent need to discover suitable methods for the treatment of cancer, such as for example multiple myeloma, including combination treatments that result in decreased side effects and that are effective at treating and controlling malignancies.

### SUMMARY OF THE INVENTION

The present invention is based on the discovery that histone deacetylase (HDAC) inhibitors, for example suberoylanilide hydroxamic acid (SAHA), can be used in combination with Bortezomib, to provide therapeutic efficacy.

The invention relates to a method for treating cancer or other disease comprising administering to a subject in need thereof an amount of an HDAC inhibitor, e.g., SAHA, and an amount of another anti-cancer agent, e.g., Bortezomib. Bortezomib is sold under the name Velcade®.

The invention further relates to pharmaceutical combinations useful for the treatment of cancer or other disease comprising an amount of an HDAC inhibitor, e.g., SAHA, and an amount of an anti-cancer agent, e.g., Bortezomib.

In particular embodiments of this invention, the combined treatments together comprise a therapeutically effective amount. In addition, the combination of the HDAC inhibitor, and anti-cancer agent, e.g. Bortezomib can provide additive or synergistic therapeutic effects.

In further embodiments, the treatment procedures are performed sequentially in any order, alternating in any order, simultaneously, or any combination thereof. In particular, the administration of an HDAC inhibitor, e.g., SAHA, and the administration of the anti-cancer agent, e.g., Bortezomib, can be performed concurrently, consecutively, or, for example, alternating concurrent and consecutive administration.

The invention further relates to methods for selectively inducing terminal differentiation, cell growth arrest, and/or apoptosis of neoplastic cells, thereby inhibiting proliferation of such cells in a subject by administering to the subject an amount of an HDAC inhibitor, e.g., SAHA, an amount of an anti-cancer agent, e.g. Bortezomib, wherein the HDAC inhibitor and Bortezomib are administered in amounts effective to induce terminal differentiation, cell growth arrest, or apoptosis of the cells.

The invention further relates to in vitro methods for selectively inducing terminal differentiation, cell growth arrest, and/or apoptosis of neoplastic cells, thereby inhibiting proliferation of such cells, by contacting the cells with an amount of an HDAC inhibitor, e.g., SAHA, an amount of an anti-cancer agent, e.g. Bortezomib, wherein the HDAC inhibitor and second (and optional third and/or fourth) anti-cancer agent are administered in amounts effective to induce terminal differentiation, cell growth arrest, or apoptosis of the cells.

In aspect of the invention, SAHA or pharmaceutically acceptable salt or hydrate thereof is administered orally.

In another aspect of the invention, Bortezomib or pharmaceutically acceptable salt or hydrate thereof is administered intravenously.

In another embodiment, SAHA or pharmaceutically acceptable salt or hydrate thereof is administered once daily at a dose of 400 mg for at least one treatment period of 7 out of 21 days.

In yet another embodiment, SAHA or pharmaceutically acceptable salt or hydrate thereof is administered once daily at a dose of 400 mg for at least one treatment period of 10 out of 21 days.

In another embodiment, SAHA or pharmaceutically acceptable salt or hydrate thereof is administered twice daily at a dose of 200 mg for at least one treatment period of 14 out of 21 days.

In another embodiment, SAHA or pharmaceutically acceptable salt or hydrate thereof is administered once daily at a dose of 400 mg for at least one treatment period of 14 out of 21 days. For these embodiments, the administration of SAHA or pharmaceutically acceptable salt or hydrate thereof is repeated for up to eight treatment periods of 21 days.

In one embodiment, Bortezomib or pharmaceutically acceptable salt or hydrate thereof is administered once daily at a dose of 0.7 mg/m² on Days 4, 8, 11 and 15 out of 21 days.

In another embodiment, Bortezomib or pharmaceutically acceptable salt or hydrate thereof is administered once daily at a dose of 0.9 mg/m² on Days 4, 8, 11 and 15 out of 21 days.

In yet another embodiment, Bortezomib or pharmaceutically acceptable salt or hydrate thereof is administered once daily at a dose of 0.9 mg/m² on Days 1, 4, 8, and 11 out of 21 days.

In yet another embodiment, Bortezomib or pharmaceutically acceptable salt or hydrate thereof is administered once daily at a dose of about 1.1 mg/m² on Days 1, 4, 8, and 11 out of 21 days.

In yet another embodiment, Bortezomib or pharmaceutically acceptable salt or hydrate thereof is administered once daily at a dose of about 1.3 mg/m² on Days 1, 4, 8, and 11 out of 21 days.

In another embodiment, the SAHA or pharmaceutically acceptable salt or hydrate thereof is administered twice daily at a dose of 200 mg, and Bortezomib or pharmaceutically acceptable salt or hydrate thereof is administered at a total daily dose of 0.7 mg/m².

In yet another embodiment, the SAHA or pharmaceutically acceptable salt or hydrate thereof is administered twice daily at a dose of 200 mg, and Bortezomib or pharmaceutically acceptable salt or hydrate thereof is administered at a total daily dose of 0.9 mg/m².

In further embodiments, SAHA or pharmaceutically acceptable salt or hydrate thereof is administered once daily at a dose of 400 mg, and Bortezomib or pharmaceutically acceptable salt or hydrate thereof is administered at a total daily dose of 0.9 mg/m².

In another embodiment, the SAHA or pharmaceutically acceptable salt or hydrate thereof is administered once daily at a dose of 400 mg, and Bortezomib or pharmaceutically acceptable salt or hydrate thereof is administered at a total daily dose of 1.1 mg/m².

In further embodiments, SAHA or pharmaceutically acceptable salt or hydrate thereof is administered once daily at a dose of 400 mg, and Bortezomib or pharmaceutically acceptable salt or hydrate thereof is administered at a total daily dose of 1.3 mg/m².

The present invention also contemplates the combination of SAHA and Bortezomib further comprising dexamethasone or a pharmaceutically acceptable salt or hydrate thereof wherein the dexamethasone or pharmaceutically acceptable salt or hydrate thereof is administered orally once daily at a dose of 20 mg on Days 1-4 and 9-12 for at least one treatment period of 21 days.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and are encompassed by the following detailed description and claims.

### BRIEF DESCRIPTION OF THE DRAWINGS

The foregoing and other objects, features and advantages of the invention will be apparent from the following more particular description of various embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows the effect of the Vorinostat/Bortezomib combination on growth of multiple myeloma cell lines.

### DETAILED DESCRIPTION OF THE INVENTION

It has been unexpectedly discovered that the combination treatment procedure that includes administration of an HDAC inhibitor, SAHA, as described herein, and Bortezomib, as described herein, can provide improved therapeutic effects. Each of the treatments (administration of an HDAC inhibitor and administration of the Bortezomib) is used to provide a therapeutically effective treatment.

The invention further relates to a method of treating cancer, in a subject in need thereof, by administering to a subject in need thereof an amount of suberoylanilide hydroxamic acid (SAHA) or a pharmaceutically acceptable salt or hydrate thereof, in a treatment procedure, and an amount of antimetabolic agent, such as Bortezomib, in another treatment procedure, wherein the amounts can comprise a therapeutically effective amount. The cancer treatment effect of SAHA and the Bortezomib can be, e.g., additive or synergistic.

In one aspect, the method comprises administering to a patient in need thereof a first amount of SAHA or a pharmaceutically acceptable salt or hydrate thereof, in a first treatment procedure, and another amount of Bortezomib. The invention further relates to pharmaceutical combinations useful for the treatment cancer or other disease. In one aspect, the pharmaceutical combination comprises a first amount of an HDAC inhibitor, e.g., SAHA or a pharmaceutically acceptable salt or hydrate thereof, and another amount of anti-cancer agents, such as Bortezomib or a pharmaceutically acceptable salt or hydrate thereof. The first and second amounts can comprise a therapeutically effective amount.

The invention further relates to methods for selectively inducing terminal differentiation, cell growth arrest, and/or apoptosis of neoplastic cells, thereby inhibiting proliferation of such cells in a subject by administering to the subject an amount of an HDAC inhibitor, e.g., SAHA, an amount of an anti-cancer agent, e.g. Bortezomib, wherein the HDAC inhibitor and Bortezomib are administered in amounts effective to induce terminal differentiation, cell growth arrest, or apoptosis of the cells.

The invention further relates to in vitro methods for selectively inducing terminal differentiation, cell growth arrest, and/or apoptosis of neoplastic cells, thereby inhibiting proliferation of such cells, by contacting the cells with an amount of an HDAC inhibitor, e.g., SAHA, an amount of an anti-cancer agent, e.g. Bortezomib, wherein the HDAC inhibitor and second (and optional third and/or fourth) anti-cancer agent are administered in amounts effective to induce terminal differentiation, cell growth arrest, or apoptosis of the cells.

The combination therapy of the invention provides a therapeutic advantage in view of the differential toxicity associated with the two treatment modalities. For example, treatment with HDAC inhibitors can lead to a particular toxicity that is not seen with the anti-cancer agent, and vice versa. As such, this differential toxicity can permit each treatment to be administered at a dose at which said toxicities do not exist or are minimal, such that together the combination therapy provides a therapeutic dose while avoiding the toxicities of each of the constituents of the combination agents. Furthermore, when the therapeutic effects achieved as a result of the combination treatment are enhanced or synergistic, for example, significantly better than additive therapeutic effects, the doses of each of the agents can be reduced even further, thus lowering the associated toxicities to an even greater extent.

### DEFINITIONS

The term “treating” in its various grammatical forms in relation to the present invention refers to preventing (i.e. chemoprevention), curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease. Because some of the inventive methods involve the physical removal of the etiological agent, the artisan will recognize that they are equally effective in situations where the inventive compound is administered prior to, or simultaneous with, exposure to the etiological agent (prophylactic treatment) and situations where the inventive compounds are administered after (even well after) exposure to the etiological agent.

Treatment of cancer, as used herein, refers to partially or totally inhibiting, delaying or preventing the progression of cancer including cancer metastasis; inhibiting, delaying or preventing the recurrence of cancer including cancer metastasis; or preventing the onset or development of cancer (chemoprevention) in a mammal, for example a human. In addition, the method of the present invention is intended for the treatment of chemoprevention of human patients with cancer. However, it is also likely that the method would be effective in the treatment of cancer in other mammals.

The “anti-cancer agents” of the invention encompass those described herein, including any pharmaceutically acceptable salts or hydrates of such agents, or any free acids, free bases, or other free forms of such agents, and as non-limiting examples: A) Polar compounds (Marks et al. (1987); Friend, C., Scher, W., Holland, J. W., and Sato, T. (1971) Proc. Natl. Acad. Sci. (USA) 68: 378-382; Tanaka, M., Levy, J., Terada, M., Breslow, R., Rifkind, R. A., and Marks, P. A. (1975) Proc. Natl. Acad. Sci. (USA) 72: 1003-1006; Reuben, R. C., Wife, R. L., Breslow, R., Rifkind, R. A., and Marks, P. A. (1976) Proc. Natl. Acad. Sci. (USA) 73: 862-866); B) Derivatives of vitamin D and retinoic acid (Abe, E., Miyaura, C., Sakagami, H., Takeda, M., Konno, K., Yamazaki, T., Yoshika, S., and Suda, T. (1981) Proc. Natl. Acad. Sci. (USA) 78: 4990-4994; Schwartz, E. L., Snoddy, J. R., Kreutter, D., Rasmussen, H., and Sartorelli, A. C. (1983) Proc. Am. Assoc. Cancer Res. 24: 18; Tanenaga, K., Hozumi, M., and Sakagami, Y. (1980) Cancer Res. 40: 914-919); C) Steroid hormones (Lotem, J. and Sachs, L. (1975) Int. J. Cancer 15: 731-740); D) Growth factors (Sachs, L. (1978) Nature (Lond.) 274: 535, Metcalf, D. (1985) Science, 229: 16-22); E) Proteases (Scher, W., Scher, B. M., and Waxman, S. (1983) Exp. Hematol. 11: 490-498; Scher, W., Scher, B. M., and Waxman, S. (1982) Biochem. & Biophys. Res. Comm. 109: 348-354); F) Tumor promoters (Huberman, E. and Callaham, M. F. (1979) Proc. Natl. Acad. Sci. (USA) 76: 1293-1297; Lottem, J. and Sachs, L. (1979) Proc. Natl. Acad. Sci. (USA) 76: 5158-5162); and G) Inhibitors of DNA or RNA synthesis (Schwartz, E. L. and Sartorelli, A. C. (1982) Cancer Res. 42: 2651-2655, Terada, M., Epner, E., Nudel, U., Salmon, J., Fibach, E., Rifkind, R. A., and Marks, P. A. (1978) Proc. Natl. Acad. Sci. (USA) 75: 2795-2799; Morin, M. J. and Sartorelli, A. C. (1984) Cancer Res. 44: 2807-2812; Schwartz, E. L., Brown, B. J., Nierenberg, M., Marsh, J. C., and Sartorelli, A. C. (1983) Cancer Res. 43: 2725-2730; Sugano, H., Furusawa, M., Kawaguchi, T., and Ikawa, Y. (1973) Bibl. Hematol. 39: 943-954; Ebert, P. S., Wars, I., and Buell, D. N. (1976) Cancer Res. 36: 1809-1813; Hayashi, M., Okabe, J., and Hozumi, M. (1979) Gann 70: 235-238).

As used herein, the term “therapeutically effective amount” is intended to qualify the combined amount of treatments in the combination therapy. The combined amount will achieve the desired biological response. In the present invention, the desired biological response is partial or total inhibition, delay or prevention of the progression of cancer including cancer metastasis; inhibition, delay or prevention of the recurrence of cancer including cancer metastasis; or the prevention of the onset or development of cancer (chemoprevention) in a mammal, for example a human.

As used herein, the terms “combination treatment”, “combination therapy”, “combined treatment,” or “combinatorial treatment”, used interchangeably, refer to a treatment of an individual with at least two different therapeutic agents. According to one aspect of the invention, the individual is treated with a first therapeutic agent, e.g., SAHA or another HDAC inhibitor as described herein. The second therapeutic agent may be another HDAC inhibitor, or may be any clinically established anti-cancer agent (such as Bortezomib) as defined herein. A combinatorial treatment may include a third or even further therapeutic agent (such as dexamethasone, as defined here). The combination treatments may be carried out consecutively or concurrently.

As recited herein, “HDAC inhibitor” (e.g., SAHA) encompasses any synthetic, recombinant, or naturally-occurring inhibitor, including any pharmaceutical salts or hydrates of such inhibitors, and any free acids, free bases, or other free forms of such inhibitors. “Hydroxamic acid derivative,” as used herein, refers to the class of histone deacetylase inhibitors that are hydroxamic acid derivatives. Specific examples of inhibitors are provided herein.

A “retinoid” or “retinoid agent” (e.g., 3-methyl TTNEB) as used herein encompasses any synthetic, recombinant, or naturally-occurring compound that binds to one or more retinoid receptors, including any pharmaceutically acceptable salts or hydrates of such agents, and any free acids, free bases, or other free forms of such agents.

A “tyrosine kinase inhibitor” (e.g., Erlotinib) encompasses any synthetic, recombinant, or naturally occurring agent that binds to or otherwise decreases the activity or levels of one or more tyrosine kinases (e.g., receptor tyrosine kinases), including any pharmaceutically acceptable salts or hydrates of such inhibitors, and any free acids, free bases, or other free forms of such inhibitors. Included are tyrosine kinase inhibitors that act on EGFR (ErbB-1; HER-1). Also included are tyrosine kinase inhibitors that act specifically on EGFR. Non-limiting examples of tyrosine kinases inhibitors are provided herein.

An “adjunctive agent” refers to any compound used to enhance the effectiveness of an anti-cancer agent or to prevent or treat conditions associated with an anti-cancer agent such as low blood counts, neutropenia, anemia, thrombocytopenia, hypercalcemia, mucositis, bruising, bleeding, toxicity, fatigue, pain, nausea, and vomiting.

“Patient” or “subject” as the terms are used herein, refer to the recipient of the treatment. Mammalian and non-mammalian patients are included. In a specific embodiment, the patient is a mammal, such as a human, canine, murine, feline, bovine, ovine, swine, or caprine. In a particular embodiment, the patient is a human.

The terms “intermittent” or “intermittently” as used herein means stopping and starting at either regular or irregular intervals.

The term “hydrate” includes but is not limited to hemihydrate, monohydrate, dihydrate, trihydrate, and the like.

### Histone Deacetylases and Histone Deacetylase Inhibitors

Histone deacetylases (HDACs) include enzymes that catalyze the removal of acetyl groups from lysine residues in the amino terminal tails of the nucleosomal core histones. As such, HDACs together with histone acetyl transferases (HATs) regulate the acetylation status of histones. Histone acetylation affects gene expression and inhibitors of HDACs, such as the hydroxamic acid-based hybrid polar compound suberoylanilide hydroxamic acid (SAHA) induce growth arrest, differentiation, and/or apoptosis of transformed cells in vitro and inhibit tumor growth in vivo.

HDACs can be divided into three classes based on structural homology. Class I HDACs (HDACs 1, 2, 3, and 8) bear similarity to the yeast RPD3 protein, are located in the nucleus and are found in complexes associated with transcriptional co-repressors. Class II HDACs (HDACs 4, 5, 6, 7 and 9) are similar to the yeast HDA1 protein, and have both nuclear and cytoplasmic subcellular localization. Both Class I and II HDACs are inhibited by hydroxamic acid-based HDAC inhibitors, such as SAHA. Class III HDACs form a structurally distant class of NAD dependent enzymes that are related to the yeast SIR2 proteins and are not inhibited by hydroxamic acid-based HDAC inhibitors.

Histone deacetylase inhibitors or HDAC inhibitors are compounds that are capable of inhibiting the deacetylation of histones in vivo, in vitro or both. As such, HDAC inhibitors inhibit the activity of at least one histone deacetylase. As a result of inhibiting the deacetylation of at least one histone, an increase in acetylated histone occurs and accumulation of acetylated histone is a suitable biological marker for assessing the activity of HDAC inhibitors. Therefore, procedures that can assay for the accumulation of acetylated histones can be used to determine the HDAC inhibitory activity of compounds of interest. It is understood that compounds that can inhibit histone deacetylase activity can also bind to other substrates and as such can inhibit other biologically active molecules such as enzymes. It is also understood that the compounds of the present invention are capable of inhibiting any of the histone deacetylases set forth above, or any other histone deacetylases.

For example, in patients receiving HDAC inhibitors, the accumulation of acetylated histones in peripheral mononuclear cells as well as in tissue treated with HDAC inhibitors can be determined against a suitable control.

HDAC inhibitory activity of a particular compound can be determined in vitro using, for example, an enzymatic assay which shows inhibition of at least one histone deacetylase. Further, determination of the accumulation of acetylated histones in cells treated with a particular composition can be determinative of the HDAC inhibitory activity of a compound.

Assays for the accumulation of acetylated histones are well known in the literature. See, for example, Marks, P. A. et al., J. Natl. Cancer Inst., 92:1210-1215, 2000, Butler, L. M. et al., Cancer Res. 60:5165-5170 (2000), Richon, V. M. et al., Proc. Natl. Acad. Sci., USA, 95:3003-3007, 1998, and Yoshida, M. et al., J. Biol. Chem., 265:17174-17179, 1990.

For example, an enzymatic assay to determine the activity of an HDAC inhibitor compound can be conducted as follows. Briefly, the effect of an HDAC inhibitor compound on affinity purified human epitope-tagged (Flag) HDAC1 can be assayed by incubating the enzyme preparation in the absence of substrate on ice for about 20 minutes with the indicated amount of inhibitor compound. Substrate ([³H]acetyl-labeled murine erythroleukemia cell-derived histone) can be added and the sample can be incubated for 20 minutes at 37° C. in a total volume of 30 μL. The reaction can then be stopped and released acetate can be extracted and the amount of radioactivity release determined by scintillation counting. An alternative assay useful for determining the activity of an HDAC inhibitor compound is the “HDAC Fluorescent Activity Assay; Drug Discovery Kit-AK-500” available from BIOMOL® Research Laboratories, Inc., Plymouth Meeting, Pa.

In vivo studies can be conducted as follows. Animals, for example, mice, can be injected intraperitoneally with an HDAC inhibitor compound. Selected tissues, for example, brain, spleen, liver etc, can be isolated at predetermined times, post administration. Histones can be isolated from tissues essentially as described by Yoshida et al., J. Biol. Chem. 265:17174-17179, 1990. Equal amounts of histones (about 1 μg) can be electrophoresed on 15% SDS-polyacrylamide gels and can be transferred to Hybond-P filters (available from Amersham). Filters can be blocked with 3% milk and can be probed with a rabbit purified polyclonal anti-acetylated histone H4 antibody (αAc-H4) and anti-acetylated histone H3 antibody (αAc-H3) (Upstate Biotechnology, Inc.). Levels of acetylated histone can be visualized using a horseradish peroxidase-conjugated goat anti-rabbit antibody (1:5000) and the SuperSignal chemiluminescent substrate (Pierce). As a loading control for the histone protein, parallel gels can be run and stained with Coomassie Blue (CB).

In addition, hydroxamic acid-based HDAC inhibitors have been shown to up regulate the expression of the p21_{WAF1} gene. The p21_{WAF1} protein is induced within 2 hours of culture with HDAC inhibitors in a variety of transformed cells using standard methods. The induction of the p21_{WAF1} gene is associated with accumulation of acetylated histones in the chromatin region of this gene. Induction of p21_{WAF1} can therefore be recognized as involved in the G1 cell cycle arrest caused by HDAC inhibitors in transformed cells.

U.S. Pat. Nos. 5,369,108, 5,932,616, 5,700,811, 6,087,367 and 6,511,990, issued to some of the present inventors, disclose compounds useful for selectively inducing terminal differentiation of neoplastic cells, which compounds have two polar end groups separated by a flexible chain of methylene groups or a by a rigid phenyl group, wherein one or both of the polar end groups is a large hydrophobic group. Some of the compounds have an additional large hydrophobic group at the same end of the molecule as the first hydrophobic group which further increases differentiation activity about 100 fold in an enzymatic assay and about 50 fold in a cell differentiation assay. Methods of synthesizing the compounds used in the methods and pharmaceutical compositions of this invention are fully described the aforementioned patents, the entire contents of which are incorporated herein by reference.

Thus, the present invention includes within its broad scope compositions comprising HDAC inhibitors which are 1) hydroxamic acid derivatives; 2) Short-Chain Fatty Acids (SCFAs); 3) cyclic tetrapeptides; 4) benzamides; 5) electrophilic ketones; and/or any other class of compounds capable of inhibiting histone deacetylases, for use in inhibiting histone deacetylase, inducing terminal differentiation, cell growth arrest and/or apoptosis in neoplastic cells, and/or inducing differentiation, cell growth arrest and/or apoptosis of tumor cells in a tumor.

Non-limiting examples of such HDAC inhibitors are set forth below. It is understood that the present invention includes any salts, crystal structures, amorphous structures, hydrates, derivatives, metabolites, stereoisomers, structural isomers, and prodrugs of the HDAC inhibitors described herein.

A. Hydroxamic Acid Derivatives such as Suberoylanilide hydroxamic acid (SAHA) (Richon et al., Proc. Natl. Acad. Sci. USA 95, 3003-3007 (1998)); m-Carboxycinnamic acid bishydroxamide (CBHA) (Richon et al., supra); Pyroxamide; Trichostatin analogues such as Trichostatin A (TSA) and Trichostatin C (Koghe et al. 1998. Biochem. Pharmacol. 56: 1359-1364); Salicylbishydroxamic acid (Andrews et al., International J Parasitology 30,761-768 (2000)); Suberoyl bishydroxamic acid (SBHA) (U.S. Pat. No. 5,608,108); Azelaic bishydroxamic acid (ABHA) (Andrews et al., supra); Azelaic-1-hydroxamate-9-anilide (AAHA) (Qiu et al., Mol. Biol. Cell 11, 2069-2083 (2000)); 6-(3-Chlorophenylureido) carpoic hydroxamic acid (3Cl-UCHA); Oxamflatin [(2E)-5-[3-[(phenylsulfonyl)amino]phenyl]-pent-2-en-4-ynohydroxamic acid] (Kim et al. Oncogene, 18: 2461 2470 (1999)); A-161906, Scriptaid (Su et al. 2000 Cancer Research, 60: 3137-3142); PXD-101 (Prolifix); LAQ-824; CHAP; MW2796 (Andrews et al., supra); MW2996 (Andrews et al., supra); or any of the hydroxamic acids disclosed in U.S. Pat. Nos. 5,369,108, 5,932,616, 5,700,811, 6,087,367, and 6,511,990.

B. Cyclic Tetrapeptides such as Trapoxin A (TPX)-cyclic tetrapeptide (cyclo-(L-phenylalanyl-L-phenylalanyl-D-pipecolinyl-L-2-amino-8-oxo-9,10-epoxy decanoyl)) (Kijima et al., J. Biol. Chem. 268, 22429-22435 (1993)); FR901228 (FK 228, depsipeptide) (Nakajima et al., Ex. Cell Res. 241, 126-133 (1998)); FR225497 cyclic tetrapeptide (H. Mori et al., PCT Application WO 00/08048 (17 Feb. 2000)); Apicidin cyclic tetrapeptide [cyclo(N—O-methyl-L-tryptophanyl-L-isoleucinyl-D-pipecolinyl-L-2-amino-8-oxodecanoyl)] (Darkin-Rattray et al., Proc. Natl. Acad. Sci. USA 93, 13143-13147 (1996)); Apicidin Ia, Apicidin Ib, Apicidin Ic, Apicidin IIa, and Apicidin IIb (P. Dulski et al., PCT Application WO 97/11366); CHAP, HC-toxin cyclic tetrapeptide (Bosch et al., Plant Cell 7, 1941-1950 (1995)); WF27082 cyclic tetrapeptide (PCT Application WO 98/48825); and Chlamydocin (Bosch et al., supra).

C. Short chain fatty acid (SCFA) derivatives such as: Sodium Butyrate (Cousens et al., J. Biol. Chem. 254, 1716-1723 (1979)); Isovalerate (McBain et al., Biochem. Pharm. 53: 1357-1368 (1997)); Valerate (McBain et al., supra); 4-Phenylbutyrate (4-PBA) (Lea and Tulsyan, Anticancer Research, 15, 879-873 (1995)); Phenylbutyrate (PB) (Wang et al., Cancer Research, 59, 2766-2799 (1999)); Propionate (McBain et al., supra); Butyramide (Lea and Tulsyan, supra); Isobutyramide (Lea and Tulsyan, supra); Phenylacetate (Lea and Tulsyan, supra); 3-Bromopropionate (Lea and Tulsyan, supra); Tributyrin (Guan et al., Cancer Research, 60, 749-755 (2000)); Valproic acid, Valproate, and Pivanex™.

D. Benzamide derivatives such as CI-994; MS-275 [N-(2-aminophenyl)-4-[N-(pyridin-3-yl methoxycarbonyl)aminomethyl]benzamide] (Saito et al., Proc. Natl. Acad. Sci. USA 96, 4592-4597 (1999)); and 3′-amino derivative of MS-275 (Saito et al., supra).

E. Electrophilic ketone derivatives such as Trifluoromethyl ketones (Frey et al, Bioorganic & Med. Chem. Lett. (2002), 12, 3443-3447; U.S. Pat. No. 6,511,990) and α-keto amides such as N-methyl-α-ketoamides.

F. Other HDAC Inhibitors such as natural products, psammaplins, and Depudecin (Kwon et al. 1998. PNAS 95: 3356-3361).

Hydroxamic acid based HDAC inhibitors include suberoylanilide hydroxamic acid (SAHA), m-carboxycinnamic acid bishydroxamate (CBHA) and pyroxamide. SAHA has been shown to bind directly in the catalytic pocket of the histone deacetylase enzyme. SAHA induces cell cycle arrest, differentiation, and/or apoptosis of transformed cells in culture and inhibits tumor growth in rodents. SAHA is effective at inducing these effects in both solid tumors and hematological cancers. It has been shown that SAHA is effective at inhibiting tumor growth in animals with no toxicity to the animal. The SAHA-induced inhibition of tumor growth is associated with an accumulation of acetylated histones in the tumor. SAHA is effective at inhibiting the development and continued growth of carcinogen-induced (N-methylnitrosourea) mammary tumors in rats. SAHA was administered to the rats in their diet over the 130 days of the study. Thus, SAHA is a nontoxic, orally active antitumor agent whose mechanism of action involves the inhibition of histone deacetylase activity.

HDAC inhibitors include those disclosed in U.S. Pat. Nos. 5,369,108, 5,932,616, 5,700,811, 6,087,367, and 6,511,990, issued to some of the present inventors disclose compounds, the entire contents of which are incorporated herein by reference, non-limiting examples of which are set forth below:

Specific HDAC inhibitors include suberoylanilide hydroxamic acid (SAHA; N-Hydroxy-N′-phenyl octanediamide), which is represented by the following structural formula:

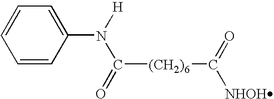

Other examples of such compounds and other HDAC inhibitors can be found in U.S. Pat. No. 5,369,108, issued on Nov. 29, 1994, U.S. Pat. No. 5,700,811, issued on Dec. 23, 1997, U.S. Pat. No. 5,773,474, issued on Jun. 30, 1998, U.S. Pat. No. 5,932,616, issued on Aug. 3, 1999 and U.S. Pat. No. 6,511,990, issued Jan. 28, 2003, all to Breslow et al.; U.S. Pat. No. 5,055,608, issued on Oct. 8, 1991, U.S. Pat. No. 5,175,191, issued on Dec. 29, 1992 and U.S. Pat. No. 5,608,108, issued on Mar. 4, 1997, all to Marks et al.; as well as Yoshida, M., et al., Bioassays 17, 423-430 (1995); Saito, A., et al., PNAS USA 96, 4592-4597, (1999); Furamai R. et al., PNAS USA 98 (1), 87-92 (2001); Komatsu, Y., et al., Cancer Res. 61(11), 4459-4466 (2001); Su, G. H., et al., Cancer Res. 60, 3137-3142 (2000); Lee, B. I. et al., Cancer Res. 61(3), 931-934; Suzuki, T., et al., J. Med. Chem. 42(15), 3001-3003 (1999); published PCT Application WO 01/18171 published on Mar. 15, 2001 to Sloan-Kettering Institute for Cancer Research and The Trustees of Columbia University; published PCT Application WO 02/246144 to Hoffmann-La Roche; published PCT Application WO 02/22577 to Novartis; published PCT Application WO 02/30879 to Prolifix; published PCT Applications WO 01/38322 (published May 31, 2001), WO 01/70675 (published on Sep. 27, 2001) and WO 00/71703 (published on Nov. 30, 2000) all to Methylgene, Inc.; published PCT Application WO 00/21979 published on Oct. 8, 1999 to Fujisawa Pharmaceutical Co., Ltd.; published PCT Application WO 98/40080 published on Mar. 11, 1998 to Beacon Laboratories, L.L.C.; and Curtin M. (Current patent status of HDAC inhibitors Expert Opin. Ther. Patents (2002) 12(9): 1375-1384 and references cited therein).

SAHA or any of the other HDACs can be synthesized according to the methods outlined in the Experimental Details Section, or according to the method set forth in U.S. Pat. Nos. 5,369,108, 5,700,811, 5,932,616 and 6,511,990, the contents of which are incorporated by reference in their entirety, or according to any other method known to a person skilled in the art.

Specific non-limiting examples of HDAC inhibitors are provided in the Table below. It should be noted that the present invention encompasses any compounds which are structurally similar to the compounds represented below, and which are capable of inhibiting histone deacetylases.

| Name | Structure |
|---|---|
| MS-275 | 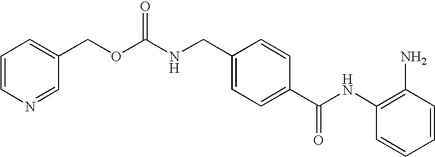 |
| DEPSIPEPTIDE | 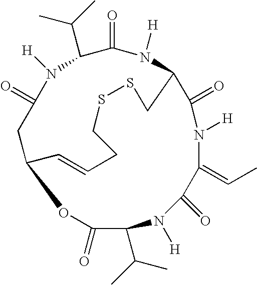 |
| CI-994 | 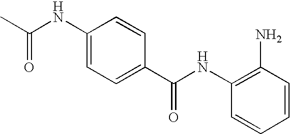 |
| Apicidin | 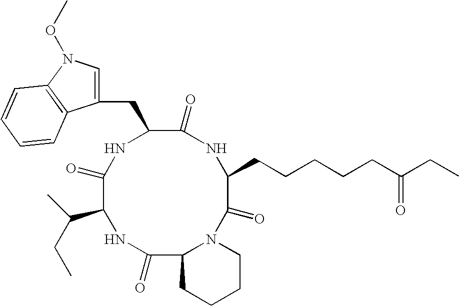 |
| A-161906 | 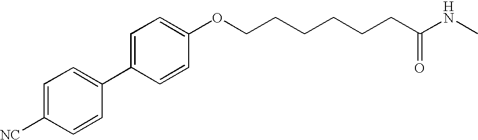 |
| Scriptaid | 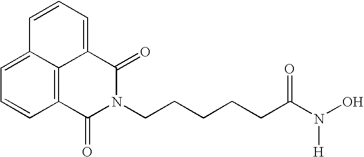 |
| PXD-101 | 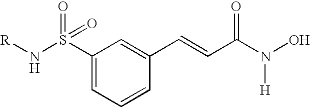 |
| CHAP | 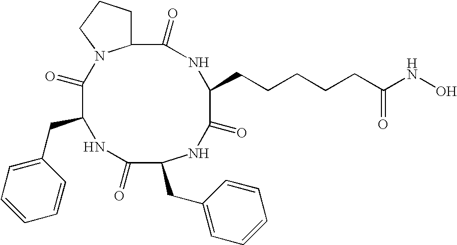 |
| LAQ-824 | 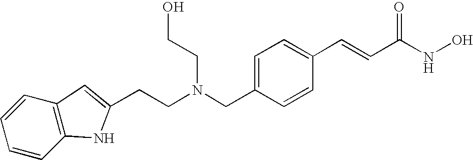 |
| Butyric Acid | 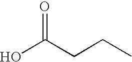 |
| Depudecin | 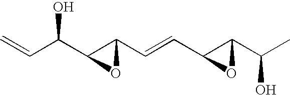 |
| Oxamflatin | 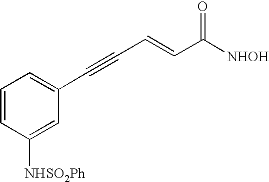 |
| Trichostatin C | 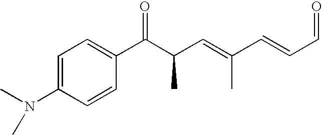 |

### Tyrosine Kinase Inhibitors and Other Therapies

Recent developments have introduced, in addition to the traditional cytotoxic and hormonal therapies used to treat cancer, additional therapies for the treatment of cancer. For example, many forms of gene therapy are undergoing preclinical or clinical trials. In addition, approaches are currently under development that are based on the inhibition of tumor vascularization (angiogenesis). The aim of this concept is to cut off the tumor from nutrition and oxygen supply provided by a newly built tumor vascular system. In addition, cancer therapy is also being attempted by the induction of terminal differentiation of the neoplastic cells. Suitable differentiation agents include the compounds disclosed in any one or more of the following references, the contents of which are incorporated by reference herein.

A) Polar compounds (Marks et al. (1987); , Friend, C., Scher, W., Holland, J. W., and Sato, T. (1971) Proc. Natl. Acad. Sci. (USA) 68: 378-382; Tanaka, M., Levy, J., Terada, M., Breslow, R., Rifkind, R. A., and Marks, P. A. (1975) Proc. Natl. Acad. Sci. (USA) 72: 1003-1006; Reuben, R. C., Wife, R. L., Breslow, R., Rifkind, R. A., and Marks, P. A. (1976) Proc. Natl. Acad. Sci. (USA) 73: 862-866); B) Derivatives of vitamin D and retinoic acid (Abe, E., Miyaura, C., Sakagami, H., Takeda, M., Konno, K., Yamazaki, T., Yoshika, S., and Suda, T. (1981) Proc. Natl. Acad. Sci. (USA) 78: 4990-4994; Schwartz, E. L., Snoddy, J. R., Kreutter, D., Rasmussen, H., and Sartorelli, A. C. (1983) Proc. Am. Assoc. Cancer Res. 24: 18; Tanenaga, K., Hozumi, M., and Sakagami, Y. (1980) Cancer Res. 40: 914-919); C) Steroid hormones (Lotem, J. and Sachs, L. (1975) Int. J. Cancer 15: 731-740); D) Growth factors (Sachs, L. (1978) Nature (Lond.) 274: 535, Metcalf, D. (1985) Science, 229: 16-22); E) Proteases (Scher, W., Scher, B. M., and Waxman, S. (1983) Exp. Hematol. 11: 490-498; Scher, W., Scher, B. M., and Waxman, S. (1982) Biochem. & Biophys. Res. Comm. 109: 348-354); F) Tumor promoters (Huberman, E. and Callaham, M. F. (1979) Proc. Natl. Acad. Sci. (USA) 76: 1293-1297; Lottem, J. and Sachs, L. (1979) Proc. Natl. Acad. Sci. (USA) 76: 5158-5162); and G) Inhibitors of DNA or RNA synthesis (Schwartz, E. L. and Sartorelli, A. C. (1982) Cancer Res. 42: 2651-2655, Terada, M., Epner, E., Nudel, U., Salmon, J., Fibach, E., Rifkind, R. A., and Marks, P. A. (1978) Proc. Natl. Acad. Sci. (USA) 75: 2795-2799; Morin, M. J. and Sartorelli, A. C. (1984) Cancer Res. 44: 2807-2812; Schwartz, E. L., Brown, B. J., Nierenberg, M., Marsh, J. C., and Sartorelli, A. C. (1983) Cancer Res. 43: 2725-2730; Sugano, H., Furusawa, M., Kawaguchi, T., and Ikawa, Y. (1973) Bibl. Hematol. 39: 943-954; Ebert, P. S., Wars, I., and Buell, D. N. (1976) Cancer Res. 36: 1809-1813; Hayashi, M., Okabe, J., and Hozumi, M. (1979) Gann 70: 235-238),

Tyrosine kinase inhibitors for use with the invention include all natural, recombinant, and synthetic agents that decrease the activity or levels of one or more tyrosine kinases (for example, receptor tyrosine kinases), e.g., EGFR (ErbB-1; HER-1), HER-2/neu (ErbB-2), HER-3 (ErbB-3), HER-4 (ErbB-4), discoidin domain receptor (DDR), ephrin receptor (EPHR), fibroblast growth factor receptor (FGFR), hepatocyte growth factor receptor (HGFR), insulin receptor (INSR), leukocytetyrosine kinase (Ltk/Alk), muscle-specific kinase (Musk), transforming growth factor receptor (e.g., TGFβ-RI and TGFβ-RII), platelet-derived growth factor receptor (PDGFR), and vascular endothelial growth factor receptor (VEGFR). Inhibitors include endogenous or modified ligands for receptor tyrosine kinases such as epidermal growth factors (e.g., EGF), nerve growth factors (e.g., NGFα, NGFβ, NGFγ), heregulins (e.g., HRGα, HRGβ), transforming growth factors (e.g., TGFα, TGFβ), epiregulins (e.g., EP), amphiregulins (e.g., AR), betacellulins (e.g., BTC), heparin-binding EGF-like growth factors (e.g., HB-EGF), neuregulins (e.g., NRG-1, NRG-2, NRG-4, NRG-4, also called glial growth factors), acetylcholine receptor-inducing activity (ARIA), and sensory motor neuron-derived growth factors (SMDGF).

Other inhibitors include DMPQ (5,7-dimethoxy-3-(4-pyridinyl)quinoline dihydrochloride), Aminogenistein (4′-amino-6-hydroxyflavone), Erbstatin analog (2,5-dihydroxymethylcinnamate, methyl 2,5-dihydroxycinnamate), Imatinib (Gleevec™, Glivec™; STI-571; 4-[(4-methyl-1-piperazinyl)methyl]-N-[4-methyl-3-[[4-(3-pyridinyl)-2-yrimidinyl]amino]-phenyl]benzamide methanesulfonate), LFM-A13 (2-Cyano-N-(2,5-dibromophenyl)-3-hydroxy-2-butenamide), PD153035 (ZM 252868; 4-[(3-bromophenyl)amino]-6,7-dimethoxyquinazoline hydrochloride), Piceatannol (trans-3,3′,4,5′-tetrahydroxystilbene, 4-[(1E)-2-(3,5-dihydroxyphenyl)ethenyl]-1,2-benzenediol), PP1 (4-amino-5-(4-methylphenyl)-7-(t-butyl)pyrazolo[3,4-d]pyrimidine), PP2 (4-amino-5-(4-chlorophenyl)-7-(t-butyl)pyrazolo[3,4,d]pyrimidine), Pertuzumab (Omnitarg™; rhuMAb2C4), SU4312 (3-[[4-(dimethylamino)phenyl]methylene]-1,3-dihydro-2H-indol-2-one), SU6656 (2,3-dihydro-N,N-dimethyl-2-oxo-3-[(4,5,6,7-tetrahydro-1H-indol-2-yl)methylene]-1H-indole-5-sulfonamide), Bevacizumab (Avastin®; rhuMAb VEGF), Semaxanib (SU5416), SU6668 (Sugen, Inc.), and ZD6126 (Angiogene Pharmaceuticals). Included are inhibitors of EGFR, e.g., Cetuximab (Erbitux; IMC-C225; MoAb C225) and Gefitinib (IRESSA™; ZD1839; ZD1839; 4-(3-chloro-4-fluoroanilino)-7-methoxy-6-(3-morpholino propoxy)quinazoline), ZD6474 (AZD6474), and EMD-72000 (Matuzumab), Panitumab (ABX-EGF; MoAb ABX-EGF), ICR-62 (MoAb ICR-62), CI-1033 (PD183805; N-[-4-[(3-Chloro-4-fluorophenyl)amino]-7-[3-(4-morpholinyl)propoxy]-6-quinazolinyl]-2-propenamide), Lapatinib (GW572016), AEE788 (pyrrolo-pyrimidine; Novartis), EKB-569 (Wyeth-Ayerst), and EXEL 7647/EXEL 09999 (EXELIS). Also included are Erlotinib and derivatives, e.g., Tarceva®; NSC 718781, CP-358774, OSI-774, R1415; N-(3-ethynylphenyl)-6,7-bis(2-methoxyethoxy)-4-quinazolinamine, as represented by the structure:

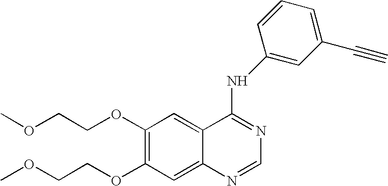

or pharmaceutically acceptable salts or hydrates thereof (e.g., methanesulfonate salt, monohydrochloride).

Agents useful for the treatment of lung cancer (e.g., NSCLC) include the above-referenced inhibitors, as well as Pemetrexed (Alimta®), Bortezomib (Velcade®), Tipifarnib, Lonafarnib, BMS214662, Prinomastat, BMS275291, Neovastat, ISIS3521 (Affinitak™; LY900003), ISIS 5132, Oblimersen (Genasenseg; G3139), and Carboxyamidotriazole (CAI) (see, e.g., Isobe T, et al., Semin. Oncol. 32:315-328, 2005).

Other agents may also be useful for use with the present invention, for example, for adjunct therapies. Such adjunctive agents can be used to enhance the effectiveness of anti-cancer agents or to prevent or treat conditions associated with anti-cancer agents such as low blood counts, neutropenia, anemia, thrombocytopenia, hypercalcemia, mucositis, bruising, bleeding, toxicity (e.g., Leucovorin), fatigue, pain, nausea, and vomiting. Agents include epoetin alpha (e.g., Procrit®, Epogen®) for stimulating red blood cell production, G-CSF (granulocyte colony-stimulating factor; filgrastim, e.g., Neupogen®) for stimulating neutrophil production, GM-CSF (granulocyte-macrophage colony-stimulating factor) for stimulating production of several white blood cells, including macrophages, and IL-11 (interleukin-11, e.g., Neumega®) for stimulating production of platelets.

Leucovorin (e.g., Leucovorin calcium, Roxane Laboratories, Inc., Columbus, Ohio) is useful as an antidote to drugs which act as folic acid antagonists. Leucovorin calcium is used to reduce the toxicity and counteract the effects of impaired methotrexate elimination and of inadvertent overdose of folic acid antagonists. Following administration, Leucovorin is absorbed and enters the general body pool of reduced folates. The increase in plasma and serum folate activity seen after administration of Leucovorin is predominantly due to 5-methyltetrahydrofolate. Leucovorin does not require reduction by the enzyme dihydrofolate reductase in order to participate in reactions utilizing folates. Leucovorin calcium is the calcium salt of N-[4-[[(2-amino-5-formyl-1,4,5,6,7,8-hexahydro-4-oxo-6-pteridinyl)methyl]amino]benzoyl]-L-glutamic acid, as represented by the structure:

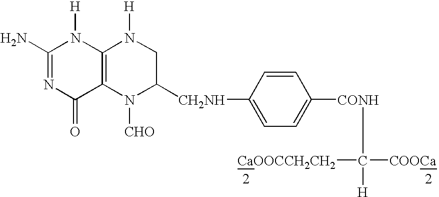

### Stereochemistry

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture.

Many of the compounds described herein can have one or more chiral centers and therefore can exist in different enantiomeric forms. If desired, a chiral carbon can be designated with an asterisk (*). When bonds to the chiral carbon are depicted as straight lines in the formulas of the invention, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the HDAC inhibitors of the present invention contain one chiral center, the compounds exist in two enantiomeric forms and the present invention includes both enantiomers and mixtures of enantiomers, such as the specific 50:50 mixture referred to as a racemic mixtures. The enantiomers can be resolved by methods known to those skilled in the art, for example by formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. For example, the enantiomeric excess can be about 60% or more, such as about 70% or more, for example about 80% or more, such as about 90% or more. In a particular embodiment when a specific absolute configuration is designated, the enantiomeric excess of depicted compounds is at least about 90%. In a more particular embodiment, the enantiomeric excess of the compounds is at least about 95%, such as at least about 97.5%, for example, at least 99% enantiomeric excess.

When a compound of the present invention has two or more chiral carbons it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers which are not mirror-images (e.g., (S,S) and (R,S)) are diastereomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

As used herein, “a,” an” and “the” include singular and plural referents unless the context clearly dictates otherwise. Thus, for example, reference to “an active agent” or “a pharmacologically active agent” includes a single active agent as well a two or more different active agents in combination, reference to “a carrier” includes mixtures of two or more carriers as well as a single carrier, and the like.

This invention is also intended to encompass pro-drugs of the HDAC inhibitors disclosed herein. A prodrug of any of the compounds can be made using well known pharmacological techniques.

This invention, in addition to the above listed compounds, is intended to encompass the use of homologs and analogs of such compounds. In this context, homologs are molecules having substantial structural similarities to the above-described compounds and analogs are molecules having substantial biological similarities regardless of structural similarities.

### Alkylating Agents

Examples of alkylating agents include, but are not limited to, bischloroethylamines (nitrogen mustards, e.g., Chlorambucil, Cyclophosphamide, Ifosfamide, Mechlorethamine, Melphalan, uracil mustard), aziridines (e.g., Thiotepa), alkyl alkone sulfonates (e.g., Busulfan), nitrosoureas (e.g., Carmustine, Lomustine, Streptozocin), nonclassic alkylating agents (Altretamine, Dacarbazine, and Procarbazine), platinum compounds (Carboplastin and Cisplatin). These compounds react with phosphate, amino, hydroxyl, sulfihydryl, carboxyl, and imidazole groups.

Cisplatin (e.g., Platinol®-AQ, Bristol-Myers Squibb Co., Princeton, N.J.) is a heavy metal complex containing a central atom of platinum surrounded by two chloride atoms, and two ammonia molecules in the cis position. The anticancer mechanism of Cisplatin is not clearly understood, but it is generally accepted that it acts through the formation of DNA adducts. Cisplatin is believed to bind to nuclear DNA and interfere with normal transcription and/or DNA replication mechanisms. Where Cisplatin-DNA adducts are not efficiently processed by cell machinery, this leads to cell death. Cells may die through apoptosis or necrosis, and both mechanisms may function within a population of tumor cells. The chemical name for Cisplatin is cis-diamminedichloroplatinum (e.g., cis-diamminedichloroplatinum (II)), as represented by the structure:

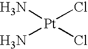

Cyclophosphamide (e.g., Cytoxan®, Baxter Healthcare Corp., Deerfield, Ill.) is chemically related to the nitrogen mustards. Cyclophosphamide is transformed to active alkylating metabolites by a mixed function microsomal oxidase system. These metabolites can interfere with the growth of rapidly proliferating malignant cells. The mechanism of action is thought to involve cross-linking of tumor cell DNA. The chemical name for Cyclophosphamide monohydrate available as Cytoxan® is 2-[bis(2-chloroethyl)amino]tetrahydro-2H-1,3,2-oxazaphosphorine 2-oxide monohydrate as represented by the structure:

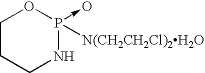

Oxaliplatin (e.g., Eloxatin™, Sanofi-Synthelabo, Inc., New York, N.Y.) is an organoplatinum complex in which the platinum atom is complexed with 1,2-diaminocyclohexane (DACH) and with an oxalate ligand as a leaving group. Oxaliplatin undergoes nonenzymatic conversion in physiologic solutions to active derivatives which form inter- and intrastrand platinum-DNA crosslinks. Crosslinks are formed between the N7 positions of two adjacent guanines (GG), adjacent adenine-guanines (AG), and guanines separated by an intervening nucleotide (GNG). These crosslinks inhibit DNA replication and transcription in cancer and non-cancer cells. The chemical name for Oxaliplatin is of cis-[(1R,2R)-1,2-cyclohexanediamine-N,N′][oxalato(2-)-O,O′]platinum, as represented by the structure:

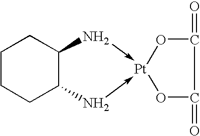

Flavopiridol (e.g., L86-8275; Alvocidib Flavopiridol (e.g., L86-8275; Alvocidib conditions, these drugs ionize and produce positively charged ion that attach to susceptible nucleic acids and proteins, leading to cell cycle arrest and/or cell death. The alkylating agents are cell cycle phase Flavopiridol (e.g., L86-8275; Alvocidib nonspecific agents because they exert their activity independently of the specific phase of the cell cycle. The nitrogen mustards and alkyl alkone sulfonates are most effective against cells in the G1 or M phase. Nitrosoureas, nitrogen mustards, and aziridines impair progression from the G1 and S phases to the M phases. Chabner and Collins eds. (1990) “Cancer Chemotherapy: Principles and Practice”, Philadelphia: JB Lippincott.

The alkylating agents are active against wide variety of neoplastic diseases, with significant activity in the treatment of leukemias and lymphomas as well as solid tumors. Clinically this group of drugs is routinely used in the treatment of acute and chronic leukemias; Hodgkin's disease; non-Hodgkin's lymphoma; multiple myeloma; primary brain tumors; carcinomas of the breast, ovaries, testes, lungs, bladder, cervix, head and neck, and malignant melanoma.

### Antibiotic Agents

Antibiotics (e.g., cytotoxic antibiotics) act by directly inhibiting DNA or RNA synthesis and are effective throughout the cell cycle. Examples of antibiotic agents include anthracyclines (e.g., Doxorubicin, Daunorubicin, Epirubicin, Idarubicin, and Anthracenedione), Mitomycin C, Bleomycin, Dactinomycin, Plicatomycin. These antibiotic agents interfere with cell growth by targeting different cellular components. For example, anthracyclines are generally believed to interfere with the action of DNA topoisomerase II in the regions of transcriptionally active DNA, which leads to DNA strand scissions.

Idarubicin (e.g., Idamycin PFS®, Pharmacia & Upjohn Co., Kalamazoo, Mich.) is a DNA-intercalating analog of daunorubicin which has an inhibitory effect on nucleic acid synthesis and interacts with the enzyme topoisomerase II. The chemical name for idarubicin hydrochloride is 5,12-naphthacenedione, 9-acetyl-7-[(3-amino-2,3,6-trideoxy-α-L-lyxo-hexopyranosyl)oxy]-7,8,9,10-tetrahydro-6,9,11-trihydroxyhydro chloride, (7S-cis) as represented by the structure:

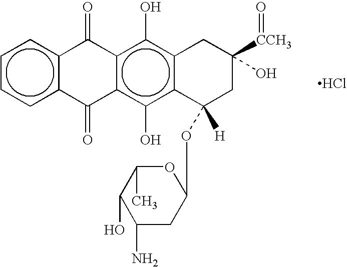

Doxorubicin (e.g., Adriamycin®, Ben Venue Laboratories, Inc., Bedford, Ohio) is a cytotoxic anthracycline antibiotic isolated from cultures of Streptomyces peucetius var. caesius. Doxorubicin binds to nucleic acids, presumably by specific intercalation of the planar anthracycline nucleus with the DNA double helix. Doxorubicin consists of a naphthacenequinone nucleus linked through a glycosidic bond at ring atom 7 to an amino sugar, daunosamine. The chemical name for Doxorubicin hydrochloride is (8S,10S)-10-[(3-Amino-2,3,6-trideoxy-α-L-lyxo-hexopyranosyl)-oxy]-8-glycoloyl-7,8,9,10-tetrahydro-6,8,11-trihydroxy-1-methoxy-5,12-naphthacenedione hydrochloride as represented by the structure:

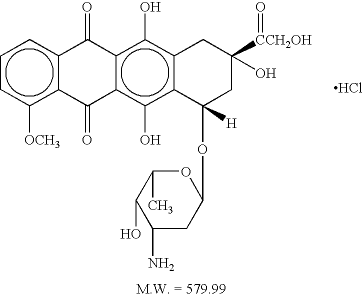

Bleomycin is generally believed to chelate iron and forms an activated complex, which then binds to bases of DNA, causing strand scissions and cell death.

The antibiotic agents have been used as therapeutics across a range of neoplastic diseases, including carcinomas of the breast, lung, stomach and thyroids, lymphomas, myelogenous leukemias, myelomas, and sarcomas.

### Antimetabolic Agents

Antimetabolic agents (i.e., antimetabolites) are a group of drugs that interfere with metabolic processes vital to the physiology and proliferation of cancer cells. Actively proliferating cancer cells require continuous synthesis of large quantities of nucleic acids, proteins, lipids, and other vital cellular constituents.

Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Antimitotic agents are included in this group. Examples of antimetabolic agents include, but are not limited to, Fluorouracil (5-FU), Floxuridine (5-FUdR), Methotrexate, Leucovorin, Hydroxyurea, Thioguanine (6-TG), Mercaptopurine (6-MP), Cytarabine, Pentostatin, Fludarabine Phosphate, Cladribine (2-CDA), Asparaginase, and Gemcitabine.

Gemcitabine (e.g., Gemzar® HCl, Eli Lilly and Co., Indianapolis, Ind.) is a nucleoside analogue that exhibits antitumor activity. Gemcitabine exhibits cell phase specificity, primarily killing cells undergoing DNA synthesis (S-phase) and also blocking the progression of cells through the G1/S-phase boundary. Gemcitabine is metabolized intracellularly by nucleoside kinases to the active diphosphate (dFdCDP) and triphosphate (dFdCTP) nucleosides. The cytotoxic effect of Gemcitabine is attributed to a combination of two actions of the diphosphate and the triphosphate nucleosides, which leads to inhibition of DNA synthesis. Gemcitabine induces internucleosomal DNA fragmentation, one of the characteristics of programmed cell death. The chemical name for Gemcitabine hydrochloride is 2′-deoxy-2′,2′-difluorocytidine monohydrochloride (β-isomer) as represented by the structure:

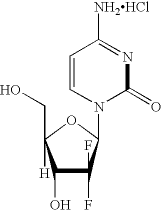

Bortezomib (e.g., Velcade®, Millennium Pharmaceuticals, Inc., Cambridge, Mass.) is a modified dipeptidyl boronic acid. Bortezomib is a reversible inhibitor of the 26S proteasome in mammalian cells. Inhibition of the 26S proteasome prevents targeted proteolysis, which can affect multiple signaling cascades within the cell. This disruption of normal homeostatic mechanisms can lead to cell death. Experiments have demonstrated that Bortezomib is cytotoxic in vitro and causes a delay in cell growth in vivo. The chemical name for Bortezomib, the monomeric boronic acid, is [(1R)-3-methyl-1-[[(2S)-1-oxo-3-phenyl-2-[(pyrazinylcarbonyl)amino]propyl]amino]butyl]boronic acid, as represented by the following structure:

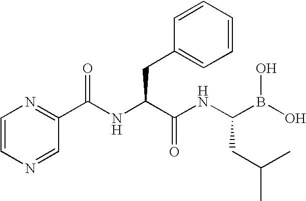

Pemetrexed (e.g., Altima®, Eli Lilly and Co., Indianapolis, Ind.) is an antifolate agent that exerts its action by disrupting folate-dependent metabolic processes essential for cell replication. In vitro studies have shown that Pemetrexed inhibits thymidylate synthase (TS), dihydrofolate reductase (DHFR), and glycinamide ribonucleotide formyltransferase (GARFT), all folate-dependent enzymes involved in the de novo biosynthesis of thymidine and purine nucleotides. Pemetrexed disodium heptahydrate has the chemical name L-glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, as represented by the structure:

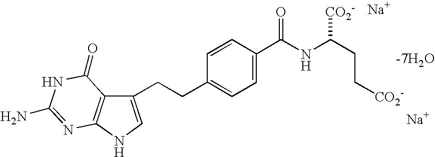

Azacitidine (e.g., Vidaza™, Pharmion Corp., Boulder, Colo.) is a pyrimidine nucleoside analog of cytidine which causes hypermethylation of DNA and direct cytotoxicity on abnormal hematopoietic cells in bone marrow. Hypermethylation may restore normal function to genes that are involved in differentiation and proliferation without causing major suppression of DNA synthesis. The cytotoxic effects of Azacitidine cause the death of rapidly dividing cells, including cells that are non longer sensitive to normal growth control mechanisms. The chemical name for Azacitidine is 4-amino-1β-D-ribofuranosyl-s-trianzin-2(1H)-one, as represented by the structure:

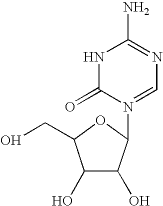

Flavopiridol (e.g., L86-8275; Alvocidib; Aventis Pharmaceuticals, Inc., Bridgewater, N.J.) is a synthetic flavone that acts as an inhibitor of the cyclin-dependent kinases (CDKs). The activation of CDKs is required for transit of the cell between the different phases of the cell cycle, including G1 to S and G2 to M. Flavopiridol has been shown to block cell cycle progression at G1-S and G2-M stages and to induce apoptosis in vitro. The chemical formula for Flavopiridol as found in Alvocidib is (−)-2-(2-chlorophenyl)-5,7-dihydroxy-8-[(3R,4S)-3-hydroxy-1-methyl-4-piperidinyl]-4H-1-benzopyran-4-one hydrochloride, as represented by the structure:

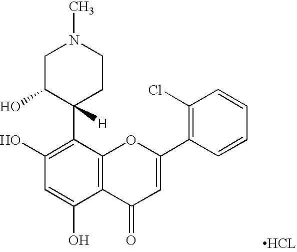

Fluorouracil (e.g., Fluorouracil Injection, Gensia Sicor Pharmaceuticals, Inc., Irvine, Calif.; Adrucil®, SP Pharmaceuticals Albuquerque, N. Mex.) is a fluorinated pyrimidine. The metabolism of fluorouracil in the anabolic pathway may block the methylation reaction of deoxyuridylic acid to thymidylic acid. In this manner, fluorouracil can interfere with the synthesis of DNA and to a lesser extent inhibits the formation of ribonucleic acid (RNA). Since DNA and RNA are essential for cell division and growth, the effect of fluorouracil may be to create a thymine deficiency which provokes unbalanced growth and death of the cell. The effects of DNA and RNA inhibition are most marked on those cells which grow more rapidly and which take up fluorouracil at a more rapid rate. The chemical formula for Fluorouracil is 5-fluoro-2,4 (1H,3H)-pyrimidinedione, as represented by the structure:

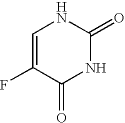

Antimetabolic agents have been widely used to treat several common forms of cancer including carcinomas of colon, rectum, breast, liver, stomach and pancreas, malignant melanoma, acute and chronic leukemia and hair cell leukemia.

### Hormonal Agents

The hormonal agents are a group of drug that regulate the growth and development of their target organs. Most of the hormonal agents are sex steroids and their derivatives and analogs thereof, such as estrogens, progestogens, anti-estrogens, androgens, anti-androgens and progestins. These hormonal agents may serve as antagonists of receptors for the sex steroids to down regulate receptor expression and transcription of vital genes. Examples of such hormonal agents are synthetic estrogens (e.g., Diethylstibestrol), antiestrogens (e.g., Tamoxifen, Toremifene, Fluoxymesterol, and Raloxifene), antiandrogens (e.g., Bicalutamide, Nilutamide, and Flutamide), aromatase inhibitors (e.g., Aminoglutethimide, Anastrozole, and Tetrazole), luteinizing hormone release hormone (LHRH) analogues, Ketoconazole, Goserelin Acetate, Leuprolide, Megestrol Acetate, and Mifepristone.

Prednisone (e.g., Deltasone®, Pharmacia & Upjohn Co., Kalamazoo, Mich.) is an adrenocortical steroid and a synthetic glucocorticoid which is readily absorbed in the gastrointestinal tract. Glucocorticoids modify the body's immune responses to diverse stimuli. Synthetic glucocorticoids are primarily used for their anti-inflammatory effects and management of leukemias and lymphomas, and other hematological disorders such as thrombocytopenia, erythroblastopenia, and anemia. The chemical name for Prednisone is pregna-1,4-diene-3,11,20-trione, 17,21-dihydroxy- (also, 1,4-pregnadiene-17α,21-diol-3,11,20-trione; 1-Cortisone; 17α,21-dihydroxy-1,4-pregnadiene-3,11,20-trione; and dehydrocortisone), as represented by the structure:

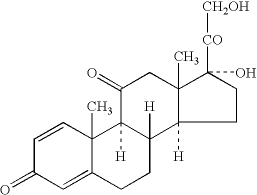

Hormonal agents are used to treat breast cancer, prostate cancer, melanoma, and meningioma. Because the major action of hormones is mediated through steroid receptors, 60% receptor-positive breast cancer responded to first-line hormonal therapy; and less than 10% of receptor-negative tumors responded. The main side effect associated with hormonal agents is flare. The frequent manifestations are an abrupt increase of bone pain, erythema around skin lesions, and induced hypercalcemia.

Specifically, progestogens are used to treat endometrial cancers, since these cancers occur in women that are exposed to high levels of oestrogen unopposed by progestogen.

Antiandrogens are used primarily for the treatment of prostate cancer, which is hormone dependent. They are used to decrease levels of testosterone, and thereby inhibit growth of the tumor.

Hormonal treatment of breast cancer involves reducing the level of oestrogen-dependent activation of oestrogen receptors in neoplastic breast cells. Anti-oestrogens act by binding to oestrogen receptors and prevent the recruitment of coactivators, thus inhibiting the oestrogen signal.

LHRH analogues are used in the treatment of prostate cancer to decrease levels of testosterone and so decrease the growth of the tumor.

Aromatase inhibitors act by inhibiting the enzyme required for hormone synthesis. In post-menopausal women, the main source of oestrogen is through the conversion of androstenedione by aromatase.

### Plant-Derived Agents

Plant-derived agents are a group of drugs that are derived from plants or modified based on the molecular structure of the agents. They inhibit cell replication by preventing the assembly of the cell's components that are essential to cell division.

Examples of plant derived agents include vinca alkaloids (e.g., Vincristine, Vinblastine, Vindesine, Vinzolidine, and Vinorelbine), podophyllotoxins (e.g., Etoposide (VP-16) and Teniposide (VM-26)), and taxanes (e.g., Paclitaxel and Docetaxel). These plant-derived agents generally act as antimitotic agents that bind to tubulin and inhibit mitosis. Podophyllotoxins such as Etoposide are believed to interfere with DNA synthesis by interacting with topoisomerase II, leading to DNA strand scission.

Vincristine (e.g., Vincristine sulfate, Gensia Sicor Pharmaceuticals, Irvine, Calif.) is an alkaloid obtained from a common flowering herb, the periwinkle plant (Vinca rosea Linn). Vincristine was originally identified as Leurocristine, and has also been referred to as LCR and VCR. The mechanism of action of Vincristine has been related to the inhibition of microtubule formation in the mitotic spindle, resulting in an arrest of dividing cells at the metaphase stage. Vincristine sulfate is vincaleukoblastine, 22-oxo-, sulfate (1:1) (salt) as represented by the structure:

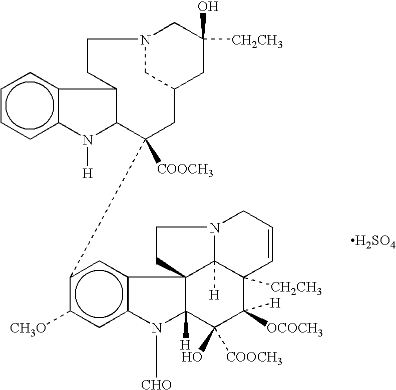

Etoposide (e.g., VePesid®, Bristol-Myers Squibb Co., Princeton, N.J., also commonly known as VP-16) is a semisynthetic derivative of podophyllotoxin. Etoposide has been shown to cause metaphase arrest and G2 arrest in mammalian cells. At high concentrations, Etoposide triggers lysis of cells entering mitosis. At low concentrations, Etoposide inhibits entry of cells into prophase. The predominant macromolecular effect of Etoposide appears to be the induction of DNA strand breaks by an interaction with DNA topoisomerase II or the formation of free radicals. Etoposide phosphate (e.g., Etopophos®, Bristol-Myers Squibb Co., Princeton, N.J.) is a water soluble ester of Etoposide. The chemical name for Etoposide phosphate is 4′-demethylepipodophyllotoxin 9-[4,6-O—(R)-ethylidene-b-D-glucopyranoside], 4′-(dihydrogen phosphate), as represented by the structure:

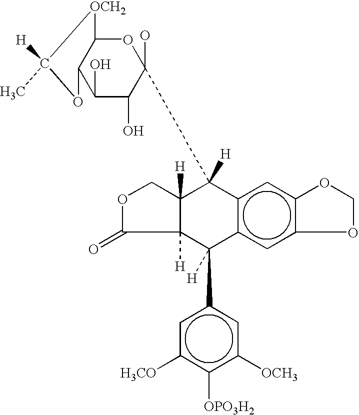

The chemical name for Etoposide is 4′-demethylepipodophyllotoxin 9-[4,6-O—(R)-ethylidene-b-D-glucopyranoside] as represented by the structure:

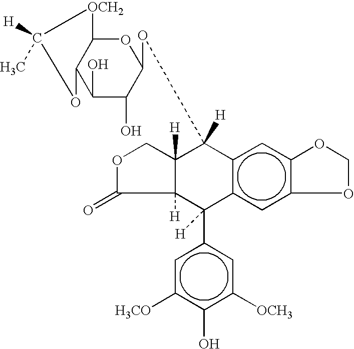

Plant-derived agents are used to treat many forms of cancer. For example, Vincristine is used in the treatment of the leukemias, Hodgkin's and non-Hodgkin's lymphoma, and the childhood tumors neuroblastoma, rhabdomyosarcoma, and Wilms' tumor. Vinblastine is used against the lymphomas, testicular cancer, renal cell carcinoma, mycosis fungoides, and Kaposi's sarcoma. Doxetaxel has shown promising activity against advanced breast cancer, non-small cell lung cancer (NSCLC), and ovarian cancer.

Etoposide is active against a wide range of neoplasms, of which small cell lung cancer, testicular cancer, and NSCLC are most responsive.

### Biologic Agents

Biologic agents are a group of biomolecules that elicit cancer/tumor regression when used alone or in combination with chemotherapy and/or radiotherapy. Examples of biologic agents include immunomodulating proteins such as cytokines, monoclonal antibodies against tumor antigens, tumor suppressor genes, and cancer vaccines.

Cytokines possess profound immunomodulatory activity. Some cytokines such as interleukin-2 (IL-2, Aldesleukin) and interferon-α (IFN-α) demonstrated antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and nonself.

Interferon-α includes more than 23 related subtypes with overlapping activities. IFN-α has demonstrated activity against many solid and hematologic malignancies, the later appearing to be particularly sensitive.

Examples of interferons include interferon-α, interferon-β (fibroblast interferon) and interferon-γ (fibroblast interferon). Examples of other cytokines include erythropoietin (Epoietin-α), granulocyte-CSF (Filgrastin), and granulocyte, macrophage-CSF (Sargramostim). Other immuno-modulating agents other than cytokines include bacillus Calmette-Guerin, levamisole, and octreotide, a long-acting octapeptide that mimics the effects of the naturally occurring hormone somatostatin.

Furthermore, the anti-cancer treatment can comprise treatment by immunotherapy with antibodies and reagents used in tumor vaccination approaches. The primary drugs in this therapy class are antibodies, alone or carrying e.g. toxins or chemotherapeutics/cytotoxics to cancer cells. Monoclonal antibodies against tumor antigens are antibodies elicited against antigens expressed by tumors, particularly tumor-specific antigens. For example, monoclonal antibody HERCEPTIN® (Trastuzumab) is raised against human epidermal growth factor receptor2 (HER2) that is overexpressed in some breast tumors including metastatic breast cancer. Overexpression of HER2 protein is associated with more aggressive disease and poorer prognosis in the clinic. HERCEPTIN® is used as a single agent for the treatment of patients with metastatic breast cancer whose tumors over express the HER2 protein.

Another example of monoclonal antibodies against tumor antigens is RITUXAN® (Rituximab) that is raised against CD20 on lymphoma cells and selectively deplete normal and malignant CD20+pre-B and mature B cells.

RITUXAN is used as single agent for the treatment of patients with relapsed or refractory low-grade or follicular, CD20+, B cell non-Hodgkin's lymphoma. MYELOTARG® (Gemtuzumab Ozogamicin) and CAMPATH® (Alemtuzumab) are further examples of monoclonal antibodies against tumor antigens that may be used.

Endostatin is a cleavage product of plasminogen used to target angiogenesis.

Tumor suppressor genes are genes that function to inhibit the cell growth and division cycles, thus preventing the development of neoplasia. Mutations in tumor suppressor genes cause the cell to ignore one or more of the components of the network of inhibitory signals, overcoming the cell cycle checkpoints and resulting in a higher rate of controlled cell growth-cancer. Examples of the tumor suppressor genes include Duc-4, NF-1, NF-2, RB, p53, WT1, BRCA1, and BRCA2.

DPC4 is involved in pancreatic cancer and participates in a cytoplasmic pathway that inhibits cell division. NF-1 codes for a protein that inhibits Ras, a cytoplasmic inhibitory protein. NF-1 is involved in neurofibroma and pheochromocytomas of the nervous system and myeloid leukemia. NF-2 encodes a nuclear protein that is involved in meningioma, schwanoma, and ependymoma of the nervous system. RB codes for the pRB protein, a nuclear protein that is a major inhibitor of cell cycle. RB is involved in retinoblastoma as well as bone, bladder, small cell lung and breast cancer. P53 codes for p53 protein that regulates cell division and can induce apoptosis. Mutation and/or inaction of p53 is found in a wide range of cancers. WTI is involved in Wilms' tumor of the kidneys. BRCA1 is involved in breast and ovarian cancer, and BRCA2 is involved in breast cancer. The tumor suppressor gene can be transferred into the tumor cells where it exerts its tumor suppressing functions.

Cancer vaccines are a group of agents that induce the body's specific immune response to tumors. Most of cancer vaccines under research and development and clinical trials are tumor-associated antigens (TAAs). TAAs are structures (i.e., proteins, enzymes, or carbohydrates) that are present on tumor cells and relatively absent or diminished on normal cells. By virtue of being fairly unique to the tumor cell, TAAs provide targets for the immune system to recognize and cause their destruction. Examples of TAAs include gangliosides (GM2), prostate specific antigen (PSA), α-fetoprotein (AFP), carcinoembryonic antigen (CEA) (produced by colon cancers and other adenocarcinomas, e.g., breast, lung, gastric, and pancreatic cancers), melanoma-associated antigens (MART-1, gap100, MAGE 1,3 tyrosinase), papillomavirus E6 and E7 fragments, whole cells or portions/lysates of autologous tumor cells and allogeneic tumor cells.

Retinoids or retinoid agents for use with the invention include all natural, recombinant, and synthetic derivatives or mimetics of vitamin A, for example, retinyl palmitate, retinoyl-beta-glucuronide (vitamin A1 beta-glucuronide), retinyl phosphate (vitamin A1 phosphate), retinyl esters, 4-oxoretinol, 4-oxoretinaldehyde, 3-dehydroretinol (vitamin A2), 11-cis-retinal (11-cis-retinaldehyde, 11-cis or neo b vitamin A1 aldehyde), 5,6-epoxyretinol (5,6-epoxy vitamin A1 alcohol), anhydroretinol (anhydro vitamin A1) and 4-ketoretinol (4-keto-vitamin A1 alcohol), all-trans retinoic acid (ATRA; Tretinoin; vitamin A acid; 3,7-dimethyl-9-(2,6,6,-trimethyl-1-cyclohenen-1-yl)-2,4,6,8-nonatetraenoic acid [CAS No. 302-79-4]), lipid formulations of all-trans retinoic acid (e.g., ATRA-IV), 9-cis retinoic acid (9-cis-RA; Alitretinoin; PanretinC; LGD1057), (e)-4-[2-(5,6,7,8-tetrahydro-2-naphthalenyl)-1-propenyl]-benzoic acid, 3-methyl-(E)-4-[2-(5,6,7,8-tetrahydro-2-naphthalenyl)-1-propenyl]-benzoic acid, Fenretinide (N-(4-hydroxyphenyl)retinamide; 4-HPR), Etretinate (2,4,6,8-nonatetraenoic acid), Acitretin (Ro 10-1670), Tazarotene (ethyl 6-[2-(4,4-dimethylthiochroman-6-yl)-ethynyl]nicotinate), Tocoretinate (9-cis-tretinoin tocoferil), Adapalene (6-[3-(1-adamantyl)-4-methoxyphenyl]-2-naphthoic acid), Motretinide (trimethylmethoxyphenyl-N-ethyl retinamide), and retinaldehyde.

Also included as retinoids are retinoid related molecules such as CD437 (also called 6-[3-(1-adamantyl)-4-hydroxphenyl]-2-naphthalene carboxylic acid and AHPN), CD2325, ST1926 ([E-3-(4′-hydroxy-3′-adamantylbiphenyl-4-yl)acrylic acid), ST1878 (methyl 2-[3-[2-[3-(2-methoxy-1,1-dimethyl-2-oxoethoxy)pheno-xy]ethoxy]phenoxy]isobutyrate), ST2307, ST1898, ST2306, ST2474, MM11453, MM002 (3-Cl-AHPC), MX2870-1, MX3350-1, MX84, and MX90-1 (Garattini et al., 2004, Curr. Pharmaceut. Design 10:433-448; Garattini and Terao, 2004, J. Chemother. 16:70-73). Included for use with the invention are retinoid agents that bind to one or more RXR. Also included are retinoid agents that bind to one or more RXR and do not bind to one or more RAR (i.e., selective binding to RXR; rexinoids), e.g., docosahexanoic acid (DHA), phytanic acid, methoprene acid, LG100268 (LG268), LG100324, LGD1057, SR11203, SR11217, SR11234, SR11236, SR11246, AGN194204 (see, e.g., Simeone and Tari, 2004, Cell Mol. Life. Sci. 61:1475-1484; Rigas and Dragnev, 2005The Oncologist 10:22-33; Ahuja et al., 2001, Mol. Pharmacol. 59:765-773; Gorgun and Foss, 2002, Blood 100:1399-1403; Bischoff et al., 1999, J. Natl. Cancer Inst. 91:2118-2123; Sun et al., 1999, Clin. Cancer Res. 5:431-437; Crow and Chandraratna, 2004, Breast Cancer Res. 6:R546-R555). Further included are derivatives of 9-cis-RA. Particularly included are 3-methyl TTNEB and related agents, e.g., Targretin®; Bexarotene; LGD1069; 4-[1-(5,6,7,8-tetrahydro-3,5,5,8,8-pentamethyl-2-naphthalenyl)ethenyl]benzoic acid, or a pharmaceutically acceptable salt or hydrate thereof.

The use of all of these approaches in combination with HDAC inhibitors, e.g. SAHA, is within the scope of the present invention.

### Other Agents

Other agents may also be useful for use with the present invention, for example, for adjunct therapies. Such adjunctive agents can be used to enhance the effectiveness of anticancer agents or to prevent or treat conditions associated with anticancer agents such as low blood counts, hypersensitivity reactions, neutropenia, anemia, thrombocytopenia, hypercalcemia, mucositis, bruising, bleeding, toxicity (e.g., Leucovorin), fatigue, pain, nausea, and vomiting. Antiemetic agents (e.g., 5-HT receptor blockers or benzodiazepines), anti-inflammatory agents (e.g., adrenocortical steroids or antihistamines), dietary supplements (e.g., folic acid), vitamins (e.g., Vitamin E, Vitamin C, Vitamin B₆, Vitamin B₁₂), and acid reducing agents (e.g., H₂ receptor blockers) can be useful for increasing patient tolerance to cancer therapy. Examples of H₂ receptor blockers include Ranitidine, Famotidine, and Cimetidine. Examples of antihistamines include Diphenhydramine, Clemastine, Chlorpheniramine, Chlorphenamine, Dimethindene maleate, and Promethazine. Examples of steroids include Dexamethasone, Hydrocortisone, and Prednisone. Other agents include growth factors such as epoetin alpha (e.g., Procrit®, Epogen®) for stimulating red blood cell production, G-CSF (granulocyte colony-stimulating factor; filgrastim, e.g., Neupogen®) for stimulating neutrophil production, GM-CSF (granulocyte-macrophage colony-stimulating factor) for stimulating production of several white blood cells, including macrophages, and IL-11 (interleukin-11, e.g., Neumega®&) for stimulating production of platelets.

Leucovorin (e.g., Leucovorin calcium, Roxane Laboratories, Inc., Columbus, Ohio; also called folinic acid, calcium folinate, citrovorum factor) can be used as an antidote to folic acid antagonists, and can also potentiate the activity of certain drugs, such as Fluorouracil. Leucovorin calcium is the calcium salt of N-[4-[[(2-amino-5-formyl-1,4,5,6,7,8-hexahydro-4-oxo-6-pteridinyl)methyl]amino]benzoyl]-L-glutamic acid.

Dexamethasone (e.g., Decadron®; Merck & Co., Inc., Whitehouse Station, N.J.) is a synthetic adrenocortical steroid that can be used as an anti-inflammatory agent to control allergic reactions, e.g., drug hypersensitivity reactions. Further, dexamethasone is used to sensitize the cells to the cytotoxic activity of anti-cancer agents. Dexamethasone tablets for oral administration comprise 9-fluoro-11-beta,17,21-trihydroxy-16-alpha-methylpregna-1,4-diene-3,20-dione, as represented by the structure:

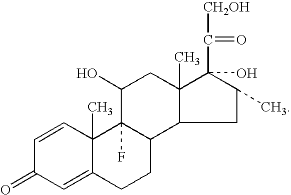

Dexamethasone phosphate for intravenous administration comprises 9-fluoro-11β,17-dihydroxy-16α-methyl-21-(phosphonooxy)pregna-1,4-diene-3,20-dione disodium salt, as represented by the structure:

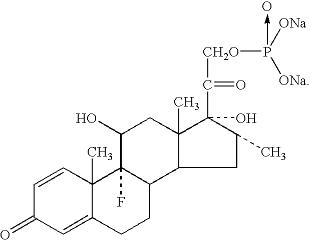

Diphenhydramine (e.g., Benadryl®; Parkedale Pharmaceuticals, Inc., Rochester, Mich.) is an antihistamine drug used for amelioration of allergic reactions. Diphenhydramine hydrochloride (e.g., Diphenhydramine HCl for injection) is 2-(diphenylmethoxy)-N,N-dimethylethylamine hydrochloride, as represented by the structure:

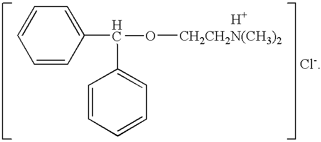

Ranitidine (e.g., Zantac®; GlaxoSmithKline, Research Triangle Park, N.C.) is a competitive inhibitor of histamine at histamine H₂-receptors, and can be used to reduce stomach acid. Ranitidine hydrochloride (e.g., tablets or injection) is N[2-[[[5-[(dimethylamino)methyl]-2-furanyl]methyl]thio]ethyl]-N′-methyl-2-nitro-1,1-ethenediamine, HCl, as represented by the structure:

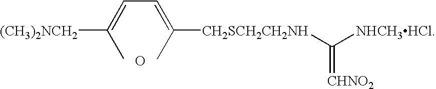

Cimetidine (e.g., Tagamet®; GlaxoSmithKline, Research Triangle Park, N.C.) is also a competitive inhibitor of histamine at histamine H2 receptors, and can be used to reduce stomach acid. Cimetidine is N″-cyano-N-methyl-N′-[2-[[(5-methyl-1H-imidazol-4-yl)methyl]thio]-ethyl]-guanidine, as represented by the structure:

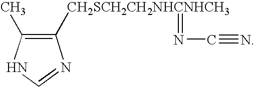

Aprepitant (e.g., EMEND®; Merck & Co., Inc.) is a substance P/neurokinin 1 (NK1) receptor antagonist and antiemetic. Aprepitant is 5-[[(2R,3S)-2-[(1R)-1-[3,5-bis(trifluoromethyl)phenyl]ethoxy]-3-(4-fluorophenyl)-4-morpholinyl]methyl]-1,2-dihydro-3H-1,2,4-triazol-3-one, as represented by the structure:

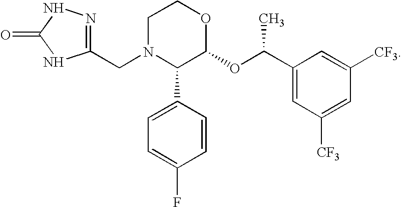

Ondansetron (e.g., Zofran®; GlaxoSmithKline, Research Triangle Park, N.C.) is a selective blocking agent of 5-HT3 serotonin receptor and antiemetic. Ondansetron hydrochloride (e.g., for injection) is (±)1,2,3,9-tetrahydro-9-methyl-3-[(2-methyl-1H-imidazol-1-yl)methyl]-4H-carbazol-4-one, monohydrochloride, dihydrate, as represented by the structure:

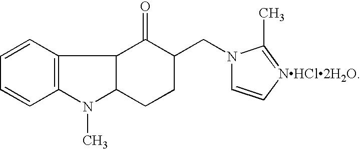

Lorazepam (e.g., Lorazepam Injection; Baxter Healthcare Corp., Deerfield, Ill.), is a benzodiazepine with anticonvulsant effects. Lorazepam is 7-chloro-5(2-chlorophenyl)-1,3-dihydro-3-hydroxy-2H-1,4-benzodiazepin-2-one, as represented by the structure:

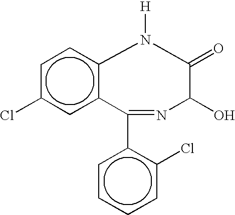

The present invention also contemplates the addition of dexamethasone to combination of SAHA and Bortezomib to increase the response rate and to sensitize the cells to the cytotoxic activity of anti-myeloma agents. In one aspect of the invention, patients who complete at least 1 cycle of treatment with vorinostat in combination with bortezomib and then experience progressive disease may be treated with dexamethasone 20 mg p.o. daily on Days 1-4, and 9-12 of each cycle along with vorinostat and bortezomib as scheduled.

### Administration of the HDAC Inhibitor

Routes of Administration

The HDAC inhibitor (e.g. SAHA), can be administered by any known administration method known to a person skilled in the art. Examples of routes of administration include but are not limited to oral, parenteral, intraperitoneal, intravenous, intraarterial, transdermal, topical, sublingual, intramuscular, rectal, transbuccal, intranasal, liposomal, via inhalation, vaginal, intraoccular, via local delivery by catheter or stent, subcutaneous, intraadiposal, intraarticular, intrathecal, or in a slow release dosage form. SAHA or any one of the HDAC inhibitors can be administered in accordance with any dose and dosing schedule that, together with the effect of the anti-cancer agent, achieves a dose effective to treat disease.

Of course, the route of administration of SAHA or any one of the other HDAC inhibitors is independent of the route of administration of the anti-cancer agent. A particular route of administration for SAHA is oral administration. Thus, in accordance with this embodiment, SAHA is administered orally, and the second agent (anti-cancer agent) can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

As examples, the HDAC inhibitors of the invention can be administered in such oral forms as tablets, capsules (each of which includes sustained release or timed release formulations), pills, powders, granules, elixirs, tinctures, suspensions, syrups, and emulsions. Likewise, the HDAC inhibitors can be administered by intravenous (e.g., bolus or infusion), intraperitoneal, subcutaneous, intramuscular, or other routes using forms well known to those of ordinary skill in the pharmaceutical arts. A particular route of administration of the HDAC inhibitor is oral administration.

The HDAC inhibitors can also be administered in the form of a depot injection or implant preparation, which may be formulated in such a manner as to permit a sustained release of the active ingredient. The active ingredient can be compressed into pellets or small cylinders and implanted subcutaneously or intramuscularly as depot injections or implants. Implants may employ inert materials such as biodegradable polymers or synthetic silicones, for example, Silastic, silicone rubber or other polymers manufactured by the Dow-Corning Corporation.

The HDAC inhibitor can also be administered in the form of liposome delivery systems, such as small unilamellar vesicles, large unilamellar vesicles and multilamellar vesicles. Liposomes can be formed from a variety of phospholipids, such as cholesterol, stearylamine, or phosphatidylcholines. Liposomal preparations of tyrosine kinase inhibitors may also be used in the methods of the invention. Liposome versions of tyrosine kinase inhibitors may be used to increase tolerance to the inhibitors.

The HDAC inhibitors can also be delivered by the use of monoclonal antibodies as individual carriers to which the compound molecules are coupled.

The HDAC inhibitors can also be prepared with soluble polymers as targetable drug carriers. Such polymers can include polyvinyl pyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the HDAC inhibitors can be prepared with biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

In a specific embodiment, the HDAC inhibitor, e.g. SAHA, is administered orally in a gelatin capsule, which can comprise excipients such as microcrystalline cellulose, croscarmellose sodium and magnesium stearate.

Dosages and Dosage Schedules

The dosage regimen utilizing the HDAC inhibitors can be selected in accordance with a variety of factors including type, species, age, weight, sex and the type of disease being treated; the severity (i.e., stage) of the disease to be treated; the route of administration; the renal and hepatic function of the patient; and the particular compound or salt thereof employed. A dosage regiment can be used, for example, to prevent, inhibit (fully or partially), or arrest the progress of the disease.

In accordance with the invention, an HDAC inhibitor (e.g., SAHA or a pharmaceutically acceptable salt or hydrate thereof) can be administered by continuous or intermittent dosages. For example, intermittent administration of an HDAC inhibitor may be administration one to six days per week or it may mean administration in cycles (e.g. daily administration for two to eight consecutive weeks, then a rest period with no administration for up to one week) or it may mean administration on alternate days. The compositions may be administered in cycles, with rest periods in between the cycles (e.g. treatment for two to eight weeks with a rest period of up to a week between treatments).

For example, SAHA or any one of the HDAC inhibitors can be administered in a total daily dose of up to 800 mg. The HDAC inhibitor can be administered once daily (QD), or divided into multiple daily doses such as twice daily (BID), and three times daily (TID). The HDAC inhibitor can be administered at a total daily dosage of up to 800 mg, e.g., 200 mg, 300 mg, 400 mg, 600 mg, or 800 mg, which can be administered in one daily dose or can be divided into multiple daily doses as described above. In specific aspects, the administration is oral.

In one embodiment, the composition is administered once daily at a dose of about 200-600 mg. In another embodiment, the composition is administered twice daily at a dose of about 200-400 mg. In another embodiment, the composition is administered twice daily at a dose of about 200-400 mg intermittently, for example three, four or five days per week. In one embodiment, the daily dose is 200 mg which can be administered once-daily, twice-daily or three-times daily. In one embodiment, the daily dose is 300 mg which can be administered once-daily, twice-daily or three-times daily. In one embodiment, the daily dose is 400 mg which can be administered once-daily, twice-daily or three-times daily.

SAHA or any one of the HDAC inhibitors can be administered in accordance with any dose and dosing schedule that, together with the effect of the anti-cancer agent, achieves a dose effective to treat cancer. The HDAC inhibitors can be administered in a total daily dose that may vary from patient to patient, and may be administered at varying dosage schedules. For example, SAHA or any of the HDAC inhibitors can be administered to the patient at a total daily dosage of between 25-4000 mg/m². In particular, SAHA or any one of the HDAC inhibitors can be administered in a total daily dose of up to 800 mg, especially by oral administration, once, twice or three times daily, continuously (every day) or intermittently (e.g., 3-5 days a week). In addition, the administration can be continuous, i.e., every day, or intermittently.

A particular treatment protocol comprises continuous administration (i.e., every day), once, twice or three times daily at a total daily dose in the range of about 200 mg to about 600 mg. Another treatment protocol comprises intermittent administration of between three to five days a week, once, twice or three times daily at a total daily dose in the range of about 200 mg to about 600 mg.

In one particular embodiment, the HDAC inhibitor is administered continuously once daily at a dose of 400 mg or twice daily at a dose of 200 mg. In another particular embodiment, the HDAC inhibitor is administered intermittently three days a week, once daily at a dose of 400 mg or twice daily at a dose of 200 mg. In another particular embodiment, the HDAC inhibitor is administered intermittently four days a week, once daily at a dose of 400 mg or twice daily at a dose of 200 mg. In another particular embodiment, the HDAC inhibitor is administered intermittently five days a week, once daily at a dose of 400 mg or twice daily at a dose of 200 mg.

In one particular embodiment, the HDAC inhibitor is administered continuously once daily at a dose of 600 mg, twice daily at a dose of 300 mg, or three times daily at a dose of 200 mg. In another particular embodiment, the HDAC inhibitor is administered intermittently three days a week, once daily at a dose of 600 mg, twice daily at a dose of 300 mg, or three times daily at a dose of 200 mg. In another particular embodiment, the HDAC inhibitor is administered intermittently four days a week, once daily at a dose of 600 mg, twice daily at a dose of 300 mg, or three times daily at a dose of 200 mg. In another particular embodiment, the HDAC inhibitor is administered intermittently five days a week, once daily at a dose of 600 mg, twice daily at a dose of 300 mg, or three times daily at a dose of 200 mg.

In addition, the HDAC inhibitor may be administered according to any of the schedules described above, consecutively for a few weeks, followed by a rest period. For example, the HDAC inhibitor may be administered according to any one of the schedules described above from two to eight weeks, followed by a rest period of one week, or twice daily at a dose of 300 mg for three to five days a week. In another particular embodiment, the HDAC inhibitor is administered three times daily for two consecutive weeks, followed by one week of rest.

In one embodiment, the composition is administered continuously (i.e., daily) or intermittently (e.g., at least 3 days per week) with a once daily dose of about 300 mg, about 400 mg, about 500 mg, about 600 mg, about 700 mg, or about 800 mg.

In another embodiment, the composition is administered once daily at a dose of about 300 mg, about 400 mg, about 500 mg, about 600 mg, about 700 mg, or about 800 mg for at least one period of 7 out of 21 days (e.g., 7 consecutive days or Days 1-7 in a 21 day cycle).

In another embodiment, the composition is administered once daily at a dose of about 400 mg, about 500 mg, or about 600 mg for at least one period of 14 out of 21 days (e.g., 14 consecutive days or Days 1-14 in a 21 day cycle).

In another embodiment, the composition is administered once daily at a dose of about 300 mg or about 400 mg for at least one period of 14 out of 28 days (e.g., 14 consecutive days or Days 1-14 of a 28 day cycle).

In another embodiment, the composition is administered once daily at a dose of about 400 mg, for example, for at least one period of 21 out of 28 days (e.g., 21 consecutive days or Days 1-21 in a 28 day cycle).

In another embodiment, the composition is administered continuously (i.e., daily) or intermittently (e.g., at least 3 days per week) with a twice daily dose of about 200 mg, about 250 mg, about 300 mg, or about 400 mg.

In another embodiment, the composition is administered twice daily at a dose of about 200 mg, about 250 mg, or about 300 mg (per dose) for at least one period of 3 out of 7 days (e.g., 3 consecutive days with dosage followed by 4 consecutive days without dosage).

In another embodiment, the composition is administered twice daily at a dose of about 200 mg, about 250 mg, or about 300 mg (per dose) for at least one period of 4 out of 7 days (e.g., 4 consecutive days with dosage followed by 3 consecutive days without dosage).

In another embodiment, the composition is administered twice daily at a dose of about 200 mg, about 250 mg, or about 300 mg (per dose) for at least one period of 5 out of 7 days (e.g., 5 consecutive days with dosage followed by 2 consecutive days without dosage).

In another embodiment, the composition is administered twice daily at a dose of about 200 mg, about 250 mg, or about 300 mg (per dose) for at least one period of 3 out of 7 days in a cycle of 21 days (e.g., 3 consecutive days or Days 1-3 for up to 3 weeks in a 21 day cycle).

In another embodiment, the composition is administered twice daily at a dose of about 200 mg, about 250 mg, or about 300 mg (per dose) for at least one period of 3 out of 7 days in a cycle of 28 days (e.g., 3 consecutive days or Days 1-3 for up to 4 weeks in a 28 day cycle).

In another embodiment, the composition is administered twice daily at a dose of about 200 mg, about 250 mg, or about 300 mg (per dose) for at least one period of 4 out of 7 days in a cycle of 21 days (e.g., 4 consecutive days or Days 1-4 for up to 3 weeks in a 21 day cycle).

In another embodiment, the composition is administered twice daily at a dose of about 200 mg, about 250 mg, or about 300 mg (per dose) for at least one period of 5 out of 7 days in a cycle of 21 days (e.g., 5 consecutive days or Days 1-5 for up to 3 weeks in a 21 day cycle).

In another embodiment, the composition is administered twice daily at a dose of about 200 mg, about 250 mg, or about 300 mg (per dose), for example, for one period of 3 out of 7 days in a cycle of 21 days (e.g., 3 consecutive days or Days 1-3 in a 21 day cycle).

In another embodiment, the composition is administered twice daily at a dose of about 200 mg, about 250 mg, or about 300 mg (per dose), for example, for at least two periods of 3 out of 7 days in a cycle of 21 days (e.g., 3 consecutive days or Days 1-3 and Days 8-10 for Week 1 and Week 2 of a 21 day cycle).

In another embodiment, the composition is administered twice daily at a dose of about 200 mg, about 250 mg, or about 300 mg (per dose), for example, for at least three periods of 3 out of 7 days in a cycle of 21 days (e.g., 3 consecutive days or Days 1-3, Days 8-10, and Days 15-17 for Week 1, Week 2, and Week 3 of a 21 day cycle).

In another embodiment, the composition is administered twice daily at a dose of about 200 mg, about 250 mg, or about 300 mg (per dose) for at least four periods of 3 out of 7 days in a cycle of 28 days (e.g., 3 consecutive days or Days 1-3, Days 8-10, Days 15-17, and Days 22-24 for Week 1, Week 2, Week 3, and Week 4 in a 28 day cycle).

In another embodiment, the composition is administered twice daily at a dose of about 300 mg (per dose), for example, for at least one period of 7 out of 14 days (e.g., 7 consecutive days or Days 1-7 in a 14 day cycle).

In another embodiment, the composition is administered twice daily at a dose of about 200 mg, about 300 mg, or about 400 mg (per dose), for example, for at least one period of 11 out of 21 days (e.g., 11 consecutive days or Days 1-11 in a 21 day cycle).

In another embodiment, the composition is administered once daily at a dose of about 200 mg, about 300 mg, or about 400 mg (per dose), for example, for at least one period of 10 out of 21 days (e.g., 10 consecutive days or Days 1-10 in a 21 day cycle).

In another embodiment, the composition is administered twice daily at a dose of about 200 mg, about 300 mg, or about 400 mg (per dose), for example, for at least one period of 10 out of 21 days (e.g., 10 consecutive days or Days 1-10 in a 21 day cycle).

In another embodiment, the composition is administered twice daily at a dose of about 200 mg, about 300 mg, or about 400 mg (per dose), for example, for at least one period of 14 out of 21 days (e.g., 14 consecutive days or Days 1-14 in a 21 day cycle).

In one preferred embodiment, SAHA or pharmaceutically acceptable salt or hydrate thereof is administered once daily at a dose of 400 mg for at least one treatment period of 7 out of 21 days. In another preferred embodiment, SAHA or pharmaceutically acceptable salt or hydrate thereof is administered once daily at a dose of 400 mg for at least one treatment period of 10 out of 21 days. In other specific embodiments, SAHA or pharmaceutically acceptable salt or hydrate thereof is administered twice daily at a dose of 200 mg for at least one treatment period of 14 out of 21 days. In further preferred embodiments, SAHA or pharmaceutically acceptable salt or hydrate thereof is administered once daily at a dose of 400 mg for at least one treatment period of 14 out of 21 days.

In addition, the HDAC inhibitor may be administered according to any of the schedules described above, consecutively for a few weeks, followed by a rest period. For example, the HDAC inhibitor may be administered according to any one of the schedules described above from two to eight weeks, followed by a rest period of one week, or twice daily at a dose of 300 mg for three to five days a week. In another particular embodiment, the HDAC inhibitor is administered three times daily for two consecutive weeks, followed by one week of rest.

Intravenously or subcutaneously, the patient would receive the HDAC inhibitor in quantities sufficient to deliver between about 3-1500 mg/m² per day, for example, about 3, 30, 60, 90, 180, 300, 600, 900, 1200 or 1500 mg/m² per day. Such quantities may be administered in a number of suitable ways, e.g. large volumes of low concentrations of HDAC inhibitor during one extended period of time or several times a day. The quantities can be administered for one or more consecutive days, intermittent days or a combination thereof per week (7 day period). Alternatively, low volumes of high concentrations of HDAC inhibitor during a short period of time, e.g. once a day for one or more days either consecutively, intermittently or a combination thereof per week (7 day period). For example, a dose of 300 mg/m² per day can be administered for 5 consecutive days for a total of 1500 mg/m² per treatment. In another dosing regimen, the number of consecutive days can also be 5, with treatment lasting for 2 or 3 consecutive weeks for a total of 3000 mg/m² and 4500 mg/m² total treatment.

Typically, an intravenous formulation may be prepared which contains a concentration of HDAC inhibitor of between about 1.0 mg/mL to about 10 mg/mL, e.g. 2.0 mg/mL, 3.0 mg/mL, 4.0 mg/mL, 5.0 mg/mL, 6.0 mg/mL, 7.0 mg/mL, 8.0 mg/mL, 9.0 mg/mL and 10 mg/mL and administered in amounts to achieve the doses described above. In one example, a sufficient volume of intravenous formulation can be administered to a patient in a day such that the total dose for the day is between about 300 and about 1500 mg/m².

Subcutaneous formulations can be prepared according to procedures well known in the art at a pH in the range between about 5 and about 12, which include suitable buffers and isotonicity agents, as described below. They can be formulated to deliver a daily dose of HDAC inhibitor in one or more daily subcutaneous administrations, e.g., one, two or three times each day.

The HDAC inhibitors can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

It is apparent to a person skilled in the art that any one or more of the specific dosages and dosage schedules of the HDAC inhibitors are also applicable to any one or more of the anti-cancer agents to be used in the combination treatment. Moreover, the specific dosage and dosage schedule of the anti-cancer agent can further vary, and the optimal dose, dosing schedule, and route of administration can be determined based upon the specific anti-cancer agent that is being used. Further, the various modes of administration, dosages, and dosing schedules described herein merely set forth specific embodiments and should not be construed as limiting the broad scope of the invention. Any permutations, variations, and combinations of the dosages and dosing schedules are included within the scope of the present invention.

### Administration of Anti-Cancer Agents

Any one or more of the specific dosages and dosage schedules of the HDAC inhibitors, is also applicable to any one or more of the anti-cancer agents to be used in the combination treatment.

Moreover, the specific dosage and dosage schedule of the anti-cancer agent can further vary, and the optimal dose, dosing schedule and route of administration will be determined based upon the specific anti-cancer agent that is being used.

Of course, the route of administration of SAHA or any one of the other HDAC inhibitors is independent of the route of administration of the anti-cancer agent. A particular route of administration for SAHA is oral administration. Thus, in accordance with this embodiment, SAHA is administered orally, and the other anti-cancer agent can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

In addition, the HDAC inhibitor and anti-cancer agent may be administered by the same mode of administration, i.e. both agents administered orally, by IV, etc. However, it is also within the scope of the present invention to administer the HDAC inhibitor by one mode of administration, e.g. oral, and to administer the anti-cancer agent by another mode of administration, e.g. IV, or any other ones of the administration modes described hereinabove.

Commonly used anti-cancer agents and daily dosages usually administered include but are not restricted to:

| | | | |
|---|---|---|---|
| Antimetabolites: | Methotrexate: | 20-40 | mg/m² i.v. |
| | Methotrexate: | 4-6 | mg/m² p.o. |
| | Methotrexate: | 12000 | mg/m² high |
| | | | dose therapy |
| | 6-Mercaptopurine: | 100 | mg/m² |
| | 6- Thioguanine: | 1-2 × 80 | mg/m² p.o. |
| | Pentostatin | 4 | mg/m² i.v. |
| | Fludarabinphosphate: | 25 | mg/m² i.v. |
| | Cladribine: | 0.14 | mg/kg BW i.v. |
| | 5-Fluorouracil | 500-2600 | mg/m² i.v. |
| | Capecitabine: | 1250 | mg/m² p.o. |
| | Cytarabin: | 200 | mg/m² i.v. |
| | Cytarabin: | 3000 | mg/m² i.v. high |
| | | | dose therapy |
| | Gemcitabine: | 800-1250 | mg/m² i.v. |
| | Hydroxyurea: | 800-4000 | mg/m² p.o. |
| | Pemetrexed | 250-500 | mg/m² i.v. |
| Antimitotic agents | Vincristine | 1.5-2 | mg/m² i.v. |
| and Plant- | Vinblastine | 4-8 | mg/m² i.v. |
| derived agents: | Vindesine | 2-3 | mg/m² i.v. |
| | Etoposide (VP16) | 100-200 | mg/m² i.v. |
| | Etoposide (VP16) | 100 | mg p.o. |
| | Teniposide (VM26) | 20-30 | mg/m² i.v. |
| | Paclitaxel (Taxol) | 175-250 | mg/m² i.v. |
| | Docetaxel (Taxotere) | 100-150 | mg/m² i.v. |
| Antibiotics: | Actinomycin D | 0.6 | mg/m2 i.v. |
| | Daunorubicin | 45-6.0 | mg/m² i.v. |
| | Doxorubicin | 45-60 | mg/m² i.v. |
| | Epirubicin | 60-80 | mg/m² i.v. |
| | Idarubicin | 10-12 | mg/m² i.v. |
| | Idarubicin | 35-50 | mg/m² p.o. |
| | Mitoxantron | 10-12 | mg/m² i.v. |
| | Bleomycin | 10-15 | mg/m² i.v., |
| | | | i.m., s.c. |
| | Mitomycin C | 10-20 | mg/² i.v. |
| | Irinotecan (CPT-11) | 350 | mg/m² i.v. |
| | Topotecan | 1.5 | mg/m² i.v. |
| Alkylating Agents: | Mustargen | 6 | mg/m² i.v. |
| | Estramustinphosphate | 150-200 | mg/m² i.v. |
| | Estramustinphosphate | 480-550 | mg/m² p.o. |
| | Melphalan | 8-10 | mg/m² i.v. |
| | Melphalan | 15 | mg/m² i.v. |
| | Chlorambucil | 3-6 | mg/m² i.v. |
| | Prednimustine | 40-100 | mg/m² p.o. |
| | Cyclophosphamide | 750-1200 | mg/m² i.v. |
| | Cyclophosphamide | 50-100 | mg/m² p.o. |
| | Ifosfamide | 1500-2000 | mg/m² i.v. |
| | Trofosfamide | 25-200 | mg/m² p.o. |
| | Busulfan | 2-6 | mg/m² p.o. |
| | Treosulfan | 5000-8000 | mg/m² i.v. |
| | Treosulfan | 750-1500 | mg/m² p.o. |
| | Thiotepa | 12-16 | mg/m² i.v. |
| | Carmustin (BCNU) | 100 | mg/m² i.v. |
| | Lomustin (CCNU) | 100-130 | mg/m² p.o. |
| | Nimustin (ACNU) | 90-100 | mg/m² i.v. |
| | Dacarbazine (OTIC) | 100-375 | mg/m² i.v. |
| | Procarbazine | 100 | mg/m² p.o. |
| | Cisplatin | 20-120 | mg/m² i.v. |
| | Carboplatin | 300-400 | mg/m² i.v. |
| Hormones, | Interferon-α | 2-10 × 10⁶ | IU/m² |
| Cytokines | Prednisone | 40-100 | mg/m² p.o. |
| and Vitamins: | Dexamethasone | 8-24 | mg p.o. |
| | G-CSF | 5-20 | μg/kg BW s.c. |
| | all-trans Retinoic Acid | 45 | mg/m² |
| | Interleukin-2 | 18 × 10⁶ | IU/m² |
| | GM-CSF | 250 | mg/m² |
| | Erythropoietin | 150 | IU/kg tiw |

The dosage regimens utilizing the anti-cancer agents described herein (or any pharmaceutically acceptable salts or hydrates of such agents, or any free acids, free bases, or other free forms of such agents) can follow the exemplary dosages herein, including those provided for HDAC inhibitors. The dosage can be selected in accordance with a variety of factors including type, species, age, weight, sex and the type of disease being treated; the severity (i.e., stage) of the disease to be treated; the route of administration; the renal and hepatic function of the patient; and the particular compound or salt thereof employed. A dosage regimen can be used, for example, to treat, to prevent, inhibit (fully or partially), or arrest the progress of the disease.

In particular embodiments, an antimetabolic agent, Bortezomib, is administered in combination with SAHA. In particular embodiments, Bortezomib can be administered (e.g., via intravenous injection of Bortezomib®) at a dose of about 0.5 to about 0.7 mg/m², about 0.7 to about 1.0 mg/m², about 1.0 to about 1.3 mg/m², or about 1.3 to about 1.5 mg/m². The dosage can be administered as a 3 to 5 second bolus, e.g., with 3 week or 5 week treatment cycles. Within each 3 week treatment cycle, Bortezomib can be administered at about 1.0 mg/m² or about 1.3 mg/m²/dose for 2 weeks (e.g., Days 1, 4, 8, and 11), followed by a 10 day rest period (e.g., Days 12 to 21). In a particular embodiment, Bortezomib can be administered at about 0.7 mg/m² on Days 1 and 4 in a 21 day treatment cycle. In other embodiments, Bortezomib can be administered at about 0.7, 0.9, 1.1, or 1.3 mg/m² on Days 1, 4, 8, and 11 in a 21 day treatment cycle Within each 5 week treatment cycle, Bortezomib can be administered at 1.3 mg/m²/dose once weekly for 4 weeks (e.g., Days 1, 8, 15, and 22), followed by a 13 day rest period (e.g., Days 23 to 35). This dosage can be continued for at least 8 treatment cycles. As particular examples, the dosage can be administered for at least eight 3 week treatment cycles, followed by at least three 5 week treatment cycles. In particular embodiments, Bortezomib is administered at a dosage of less than 3.0 mg/m². For extended therapy of more than 8 cycles, Bortezomib may be administered on the above schedule or on a maintenance schedule of once weekly for 4 weeks (e.g., Days 1, 8, 15, and 22), followed by a 13 day rest period (e.g., Days 23 to 35). In particular embodiments, at least 72 hours elapse between consecutive doses of Bortezomib. Alternatively, Bortezomib can be administered at a dose of about 0.7 mg/m², e.g., once per week. Specifically, Bortezomib can be co-administered with one or more other anti-cancer agents, e.g., SAHA. As examples, SAHA (e.g., Vorinostat) can be administered at a total daily dose of up to 400 mg, and Bortezomib can be administered at a total daily dose at a total daily dose of up to 0.7, 0.9, 1.1, or 1.3 mg/m².

In preferred embodiments, Bortezomib or pharmaceutically acceptable salt or hydrate thereof is administered once daily at a dose of 0.7 mg/m² on Days 4, 8, 11 and 15 out of 21 days. In other preferred embodiments, Bortezomib or pharmaceutically acceptable salt or hydrate thereof is administered once daily at a dose of 0.9 mg/m² on Days 4, 8, 11 and 15 out of 21 days. In other preferred embodiments, Bortezomib or pharmaceutically acceptable salt or hydrate thereof is administered once daily at a dose of 0.9 mg/m² on Days 1, 4, 8, and 11 out of 21 days.

In other preferred embodiments, Bortezomib is administered once daily at a dose of about 1.1 mg/m² on Days 1, 4, 8, and 11 out of 21 days. In further preferred embodiments, Bortezomib or pharmaceutically acceptable salt or hydrate thereof is administered once daily at a dose of about 1.3 mg/m² on Days 1, 4, 8, and 11 out of 21 days.

### Combination Administration

In accordance with the invention, HDAC inhibitors and anti-cancer agents can be used in the treatment of a wide variety of cancers, including but not limited to solid tumors (e.g., tumors of the head and neck, lung, breast, colon, prostate, bladder, rectum, brain, gastric tissue, bone, ovary, thyroid, or endometrium), hematological malignancies (e.g., leukemias, lymphomas, myelomas), carcinomas (e.g. bladder carcinoma, renal carcinoma, breast carcinoma, colorectal carcinoma), neuroblastoma, or melanoma. Non-limiting examples of these cancers include diffuse large B-cell lymphoma (DLBCL), T-cell lymphomas or leukemias, e.g., cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), as well as acute lymphocytic leukemia, acute nonlymphocytic leukemia, acute myeloid leukemia, chronic lymphocytic leukemia, chronic myelogenous leukemia, Hodgkin's disease, non-Hodgkin's lymphoma, myeloma, multiple myeloma, mesothelioma, childhood solid tumors, brain neuroblastoma, retinoblastoma, glioma, Wilms' tumor, bone cancer and soft-tissue sarcomas, common solid tumors of adults such as head and neck cancers (e.g., oral, laryngeal and esophageal), genitourinary cancers (e.g., prostate, bladder, renal, uterine, ovarian, testicular, rectal, and colon), lung cancer (e.g., small cell carcinoma and non-small cell lung carcinoma, including squamous cell carcinoma and adenocarcinoma), breast cancer, pancreatic cancer, melanoma and other skin cancers, basal cell carcinoma, metastatic skin carcinoma, squamous cell carcinoma of both ulcerating and papillary type, stomach cancer, brain cancer, liver cancer, adrenal cancer, kidney cancer, thyroid cancer, medullary carcinoma, osteosarcoma, soft-tissue sarcoma, Ewing's sarcoma, veticulum cell sarcoma, and Kaposi's sarcoma. Also included are pediatric forms of any of the cancers described herein.

Cutaneous T-cell lymphomas and peripheral T-cell lymphomas are forms of non-Hodgkin's lymphoma. Cutaneous T-cell lymphomas are a group of lymphoproliferative disorders characterized by localization of malignant T lymphocytes to the skin at presentation. CTCL frequently involves the skin, bloodstream, regional lymph nodes, and spleen. Mycosis fungoides (MF), the most common and indolent form of CTCL, is characterized by patches, plaques or tumors containing epidermotropic CD4⁺CD45RO⁺ helper/memory T cells. MF may evolve into a leukemic variant, Sézary syndrome (SS), or transform to large cell lymphoma. The condition causes severe skin itching, pain and edema. Currently, CTCL is treated topically with steroids, photochemotherapy and chemotherapy, as well as radiotherapy. Peripheral T-cell lymphomas originate from mature or peripheral (not central or thymic) T-cell lymphocytes as a clonal proliferation from a single T-cell and are usually either predominantly nodal or extranodal tumors. They have T-cell lymphocyte cell-surface markers and clonal arrangements of the T-cell receptor genes.

Approximately 16,000 to 20,000 people in the U.S. are affected by either CTCL or PTCL. These diseases are highly symptomatic. Patches, plaques and tumors are the clinical names of the different presentations. Patches are usually flat, possibly scaly and look like a “rash.” Mycosis fungoides patches are often mistaken for eczema, psoriasis or non-specific dermatitis until a proper diagnosis of mycosis fungoides is made. Plaques are thicker, raised lesions. Tumors are raised “bumps” which may or may not ulcerate. A common characteristic is itching or pruritus, although many patients do not experience itching. It is possible to have one or all three of these phases. For most patients, existing treatments are palliative but not curative.

Lung cancer remains the leading cause of cancer-related mortality in the United States and 30% to 40% of newly diagnosed patients with non-small cell lung cancer present with regionally advanced and unresectable stage III disease (Jemal A et al. CA Cancer J. Clin. 2004; 54:8-29; Dubey and Schiller The Oncologist 2005; 10:282-291; Socinski M A Semin Oncol. 2005 32(2 Suppl 3):S114-8). The median survival time of patients with stage 1V disease treated with standard chemotherapy regimens is approximately 8-11 months (Schiller J H et al. N. Engl. J. Med. 2002; 346:92-98; Fossella F et al. J. Clin. Oncol. 2003; 21:3016-3024). In the relapsed setting, the median survival time with single-agent therapy is approximately 5-7 months, and time to progression is merely 8-10 weeks (Shepherd F A et al. J. Clin. Oncol. 2000; 18:2095-2103; Fossella F V et al. J. Clin. Oncol. 2000; 18:2354-2362).

Non-small cell lung cancer (NSCLC) accounts for approximately 85% of all lung cancer cases. The majority of patients with NSCLC present with advanced disease, and this aggressive tumor is associated with a poor prognosis. The 5-year survival rate for patients with advanced (stage IIIB/IV) NSCLC is <5% (Ginsberg R J et al. In: Cancer: Principles and Practice of Oncology, DeVita V T Jr, Hellman S, Rosenberg S A, eds., 6th Edition, Philadelphia: Lippincott Williams and Wilkins, 2001:925-983). Treatment for NSCLC has been palliative, with the goals of improving symptoms and prolonging survival. Currently, platinum-based regimens are the standard of care for patients with advanced NSCLC (reviewed in Stewart D J Oncologist 2004; 9 Suppl 6:43-52). Yet, these regimens are associated with severe and often cumulative hematologic and nonhematologic toxicities, limiting dose intensity. Therefore, novel treatments and combination regimens are needed to improve the outcome for these patients.

Diffuse large B-cell lymphoma (DLBCL) is the most common B-cell non-Hodgkin's lymphoma (NHL) in the WHO (World Health Organization) classification and constitutes 30 to 40% of adult non-Hodgkin lymphomas in western countries. The standard first-line treatment is combination chemotherapy or chemotherapy with anti-CD20 antibody (Rituximab). Because of the high cost and lack of insurance coverage in many countries, it is estimated that Rituximab can only be afforded in a small percentage of NHL patients. The standard second line treatment is peripheral stem cell transplantation. This procedure is performed in a select number of cancer centers, so it is not an treatment option for most patients. The EPOCH regimen (Etoposide, Prednisone, Vincristine, Cyclophosphamide, Doxorubicin) for DLBCL has proven activity as salvage therapy, however, it rarely provide long-lasting remissions when used as a single modality.

Multiple myeloma is characterized by the neoplastic proliferation of a single clone of plasma cells engaged in the production of a monoclonal immunoglobulin (Kyle, Multiple Myeloma and Other Plasma Cell Disorders in Hematology: Basic Principles and Practice. Second edition. 1995). Although multiple myeloma cells are initially responsive to radiotherapy and chemotherapy, durable complete responses are rare and virtually all patients who respond initially ultimately relapse and die from the disease. To date, conventional treatment approaches have not resulted in long-term disease-free survival, which highlights the importance of developing new drug treatment for this incurable disease (NCCN Proceedings. Oncology. November 1998).

According to the National Cancer Institute, head and neck cancers account for three percent of all cancers in the U.S. Most head and neck cancers originate in the squamous cells lining the structures found in the head and neck, and are often referred to as squamous cell carcinomas of the head and neck (SCCHN). Some head and neck cancers originate in other types of cells, such as glandular cells. Head and neck cancers that originate in glandular cells are called adenocarcinomas. Head and neck cancers are further defined by the area in which they begin, such as the oral cavity, nasal cavity, larynx, pharynx, salivary glands, and lymph nodes of the upper part of the neck. It is estimated that 38,000 people in the U.S. developed head and neck cancer 2002. Approximately 60% of patients present with locally advanced disease. Only 30% of these patients achieve long-term remission after treatment with surgery and/or radiation. For patients with recurrent and/or metastatic disease, the median survival is approximately six months.

In various aspects of the invention, the treatment procedures are performed sequentially in any order, simultaneously, or a combination thereof. For example, the first treatment procedure, e.g., administration of an HDAC inhibitor, can take place prior to the second treatment procedure, e.g., the anti-cancer agent, after the second treatment with the anticancer agent, at the same time as the second treatment with the anticancer agent, or a combination thereof.

In one aspect of the invention, a total treatment period can be decided for the HDAC inhibitor. The anti-cancer agent can be administered prior to onset of treatment with the HDAC inhibitor or following treatment with the HDAC inhibitor. In addition, the anti-cancer agent can be administered during the period of HDAC inhibitor administration but does not need to occur over the entire HDAC inhibitor treatment period. Similarly, the HDAC inhibitor can be administered prior to onset of treatment with the anti-cancer agent or following treatment with the anti-cancer agent. In addition, the HDAC inhibitor can be administered during the period of anti-cancer agent administration but does not need to occur over the entire anti-cancer agent treatment period. Alternatively, the treatment regimen includes pre-treatment with one agent, either the HDAC inhibitor or the anti-cancer agent, followed by the addition of the other agent(s) for the duration of the treatment period.

In a particular embodiment, the combination of the HDAC inhibitor and anti-cancer agent is additive, i.e., the combination treatment regimen produces a result that is the additive effect of each constituent when it is administered alone. In accordance with this embodiment, the amount of HDAC inhibitor and the amount of the anti-cancer together constitute an effective amount to treat cancer.

In another embodiment, the combination of the HDAC inhibitor and anti-cancer agent is considered therapeutically synergistic when the combination treatment regimen produces a significantly better anti-cancer result (e.g., cell growth arrest, apoptosis, induction of differentiation, cell death) than the additive effects of each constituent when it is administered alone at a therapeutic dose. Standard statistical analysis can be employed to determine when the results are significantly better. For example, a Mann-Whitney Test or some other generally accepted statistical analysis can be employed.

In one particular embodiment of the present invention, the HDAC inhibitor and the anticancer agent Bortezomib can be administered in further combination with an additional HDAC inhibitor. In another particular embodiment of the present invention, the HDAC inhibitor and the anticancer agent Bortezomib can be administered in further combination with an alkylating agent. In another particular embodiment of the present invention, the HDAC inhibitor and the anticancer agent Bortezomib can be administered in further combination with an antibiotic agent. In another particular embodiment of the present invention, the HDAC inhibitor and the anticancer agent Bortezomib can be administered in further combination with an antimetabolic agent. In another particular embodiment of the present invention, the HDAC inhibitor and the anticancer agent Bortezomib can be administered in further combination with a hormonal agent. In another particular embodiment of the present invention, the HDAC inhibitor and the anticancer agent Bortezomib can be administered in further combination with a plant-derived agent. In another particular embodiment of the present invention, the HDAC inhibitor and the anticancer agent Bortezomib can be administered in further combination with an anti-angiogenic agent. In another particular embodiment of the present invention, the HDAC inhibitor and the anticancer agent Bortezomib can be administered in further combination with a differentiation inducing agent.

In another particular embodiment of the present invention, the HDAC inhibitor and the anticancer agent Bortezomib can be administered in further combination with a cell growth arrest inducing agent. In another particular embodiment of the present invention, the HDAC inhibitor and the anticancer agent Bortezomib can be administered in further combination with an apoptosis inducing agent. In another particular embodiment of the present invention, the HDAC inhibitor and the anticancer agent Bortezomib can be administered in further combination with a cytotoxic agent. In another particular embodiment of the present invention, the HDAC inhibitor and the anticancer agent Bortezomib can be administered in further combination with a tyrosine kinase inhibitor. In another particular embodiment of the present invention, the HDAC inhibitor and the anticancer agent Bortezomib can be administered in further combination with an adjunctive agent. In another particular embodiment of the present invention, the HDAC inhibitor and the anticancer agent Bortezomib can be administered in further combination with a biologic agent. In another particular embodiment of the present invention, the HDAC inhibitor and the anticancer agent Bortezomib can be administered in further combination with any combination of an additional HDAC inhibitor, an alkylating agent, an antibiotic agent, an antimetabolic agent, a hormonal agent, a plant-derived agent, an anti-angiogenic agent, a differentiation inducing agent, a cell growth arrest inducing agent, an apoptosis inducing agent, a cytotoxic agent, a retinoid agent, a tyrosine kinase inhibitor, an adjunctive agent, or a biologic agent.

The combination therapy can act through the induction of cancer cell differentiation, cell growth arrest, and/or apoptosis. The combination of therapy is particularly advantageous, since the dosage of each agent in a combination therapy can be reduced as compared to monotherapy with the agent, while still achieving an overall anti-tumor effect.

In preferred embodiment of the present invention, the HDAC inhibitor can be administered in combination with an antimetabolic agent. Specifically, in one preferred embodiment, SAHA or pharmaceutically acceptable salt or hydrate thereof is administered twice daily at a dose of 200 mg, and Bortezomib or pharmaceutically acceptable salt or hydrate thereof is administered at a total daily dose of 0.7 mg/m². In further specific embodiments, SAHA or pharmaceutically acceptable salt or hydrate thereof is administered twice daily at a dose of 200 mg, and Bortezomib or pharmaceutically acceptable salt or hydrate thereof is administered at a total daily dose of 0.9 mg/m². In other specific embodiments, SAHA or pharmaceutically acceptable salt or hydrate thereof is administered once daily at a dose of 400 mg, and Bortezomib or pharmaceutically acceptable salt or hydrate thereof is administered at a total daily dose of 0.9 mg/m². In other specific embodiments, SAHA or pharmaceutically acceptable salt or hydrate thereof is administered once daily at a dose of 400 mg, and Bortezomib or pharmaceutically acceptable salt or hydrate thereof is administered at a total daily dose of 1.1 mg/m².

In further specific embodiments, SAHA or pharmaceutically acceptable salt or hydrate thereof is administered once daily at a dose of 400 mg, and Bortezomib or pharmaceutically acceptable salt or hydrate thereof is administered at a total daily dose of 1.3 mg/m².

### Pharmaceutical Compositions

As described above, the compositions comprising the HDAC inhibitor and/or the anti-cancer agent can be formulated in any dosage form suitable for oral, parenteral, intraperitoneal, intravenous, intraarterial, transdermal, sublingual, intramuscular, rectal, transbuccal, intranasal, liposomal, via inhalation, vaginal, or intraocular administration, for administration via local delivery by catheter or stent, or for subcutaneous, intraadiposal, intraarticular, intrathecal administration, or for administration in a slow release dosage form.

The HDAC inhibitor and the anti-cancer agent can be formulated in the same formulation for simultaneous administration, or they can be in two separate dosage forms, which may be administered simultaneously or sequentially as described above.

The invention also encompasses pharmaceutical compositions comprising pharmaceutically acceptable salts of the HDAC inhibitors and/or the anti-cancer agents.

Suitable pharmaceutically acceptable salts of the compounds described herein and suitable for use in the method of the invention, are conventional non-toxic salts and can include a salt with a base or an acid addition salt such as a salt with an inorganic base, for example, an alkali metal salt (e.g., lithium salt, sodium salt, potassium salt, etc.), an alkaline earth metal salt (e.g., calcium salt, magnesium salt, etc.), an ammonium salt; a salt with an organic base, for example, an organic amine salt (e.g., triethylamine salt, pyridine salt, picoline salt, ethanolamine salt, triethanolamine salt, dicyclohexylamine salt, N,N′-dibenzylethylenediamine salt, etc.) etc.; an inorganic acid addition salt (e.g., hydrochloride, hydrobromide, sulfate, phosphate, etc.); an organic carboxylic or sulfonic acid addition salt (e.g., formate, acetate, trifluoroacetate, maleate, tartrate, methanesulfonate, benzenesulfonate, p-toluenesulfonate, etc.); a salt with a basic or acidic amino acid (e.g., arginine, aspartic acid, glutamic acid, etc.) and the like.

The invention also encompasses pharmaceutical compositions comprising hydrates of the HDAC inhibitors and/or the anti-cancer agents.

In addition, this invention also encompasses pharmaceutical compositions comprising any solid or liquid physical form of SAHA or any of the other HDAC inhibitors. For example, the HDAC inhibitors can be in a crystalline form, in amorphous form, and have any particle size. The HDAC inhibitor particles may be micronized, or may be agglomerated, particulate granules, powders, oils, oily suspensions or any other form of solid or liquid physical form.

For oral administration, the pharmaceutical compositions can be liquid or solid. Suitable solid oral formulations include tablets, capsules, pills, granules, pellets, and the like. Suitable liquid oral formulations include solutions, suspensions, dispersions, emulsions, oils, and the like.

Any inert excipient that is commonly used as a carrier or diluent may be used in the formulations of the present invention, such as for example, a gum, a starch, a sugar, a cellulosic material, an acrylate, or mixtures thereof. The compositions may further comprise a disintegrating agent and a lubricant, and in addition may comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof. Furthermore, the compositions of the present invention may be in the form of controlled release or immediate release formulations.

The HDAC inhibitors can be administered as active ingredients in admixture with suitable pharmaceutical diluents, excipients or carriers (collectively referred to herein as “carrier” materials or “pharmaceutically acceptable carriers”) suitably selected with respect to the intended form of administration. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions, or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide.

Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), a glidant (e.g., colloidal silicon dioxide), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The preparation of pharmaceutical compositions that contain an active component is well understood in the art, for example, by mixing, granulating, or tablet-forming processes. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic, or oily solutions and the like as detailed above.

The amount of the compound administered to the patient is less than an amount that would cause toxicity in the patient. In the certain embodiments, the amount of the compound that is administered to the patient is less than the amount that causes a concentration of the compound in the patient's plasma equal to or exceeding the toxic level of the compound. In particular embodiments, the concentration of the compound in the patient's plasma is maintained at about 10 nM. In another embodiment, the concentration of the compound in the patient's plasma is maintained at about 25 nM. In another embodiment, the concentration of the compound in the patient's plasma is maintained at about 50 nM. In another embodiment, the concentration of the compound in the patient's plasma is maintained at about 100 nM. In another embodiment, the concentration of the compound in the patient's plasma is maintained at about 500 nM. In another embodiment, the concentration of the compound in the patient's plasma is maintained at about 1,000 nM. In another embodiment, the concentration of the compound in the patient's plasma is maintained at about 2,500 nM. In another embodiment, the concentration of the compound in the patient's plasma is maintained at about 5,000 nM. The optimal amount of the compound that should be administered to the patient in the practice of the present invention will depend on the particular compound used and the type of cancer being treated.

The percentage of the active ingredient and various excipients in the formulations may vary. For example, the composition may comprise between 20 and 90%, or specifically between 50-70% by weight of the active agent.

For IV administration, Glucuronic acid, L-lactic acid, acetic acid, citric acid or any pharmaceutically acceptable acid/conjugate base with reasonable buffering capacity in the pH range acceptable for intravenous administration can be used as buffers. Sodium chloride solution wherein the pH has been adjusted to the desired range with either acid or base, for example, hydrochloric acid or sodium hydroxide, can also be employed. Typically, a pH range for the intravenous formulation can be in the range of from about 5 to about 12. A particular pH range for intravenous formulation comprising an HDAC inhibitor, wherein the HDAC inhibitor has a hydroxamic acid moiety, can be about 9 to about 12.

Subcutaneous formulations can be prepared according to procedures well known in the art at a pH in the range between about 5 and about 12, which include suitable buffers and isotonicity agents. They can be formulated to deliver a daily dose of the active agent in one or more daily subcutaneous administrations. The choice of appropriate buffer and pH of a formulation, depending on solubility of the HDAC inhibitor to be administered, is readily made by a person having ordinary skill in the art. Sodium chloride solution wherein the pH has been adjusted to the desired range with either acid or base, for example, hydrochloric acid or sodium hydroxide, can also be employed in the subcutaneous formulation. Typically, a pH range for the subcutaneous formulation can be in the range of from about 5 to about 12. A particular pH range for subcutaneous formulation of an HDAC inhibitor a hydroxamic acid moiety can be about 9 to about 12.

The compositions of the present invention can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

The present invention also provides in-vitro methods for selectively inducing terminal differentiation, cell growth arrest or apoptosis of neoplastic cells, thereby inhibiting proliferation of such cells, by contacting the cells with a first amount of suberoylanilide hydroxamic acid (SAHA) or a pharmaceutically acceptable salt or hydrate thereof, and a second amount of an anti-cancer agent, wherein the first and second amounts together comprise an amount effective to induce terminal differentiation, cell growth arrest or apoptosis of the cells.

Although the methods of the present invention can be practiced in vitro, it is contemplated that a particular embodiment for the methods of selectively inducing terminal differentiation, cell growth arrest or apoptosis of neoplastic cells will comprise contacting the cells in vivo, i.e., by administering the compounds to a subject harboring neoplastic cells or tumor cells in need of treatment.

As such, the present invention also provides methods for selectively inducing terminal differentiation, cell growth arrest and/or apoptosis of neoplastic cells, thereby inhibiting proliferation of such cells in a subject by administering to the subject a first amount of suberoylanilide hydroxamic acid (SAHA) or a pharmaceutically acceptable salt or hydrate thereof, in a first treatment procedure, and a second amount of an anti-cancer agent in a second treatment procedure, wherein the first and second amounts together comprise an amount effective to induce terminal differentiation, cell growth arrest or apoptosis of the cells.

The invention is illustrated in the examples that follow. This section is set forth to aid in an understanding of the invention but is not intended to, and should not be construed to limit in any way the invention as set forth in the claims which follow thereafter.

### EXAMPLES

The examples are presented in order to more fully illustrate the various embodiments of the invention. These examples should in no way be construed as limiting the scope of the invention recited in the appended claims.

### Example 1

### Synthesis of SAHA

SAHA can be synthesized according to the method outlined below, or according to the method set forth in U.S. Pat. No. 5,369,108, the contents of which are incorporated by reference in their entirety, or according to any other method.

In a 22 L flask was placed 3,500 g (20.09 moles) of suberic acid, and the acid melted with heat. The temperature was raised to 175° C., and then 2,040 g (21.92 moles) of aniline was added. The temperature was raised to 190° C. and held at that temperature for 20 minutes. The melt was poured into a Nalgene tank that contained 4,017 g of potassium hydroxide dissolved in 50 L of water. The mixture was stirred for 20 minutes following the addition of the melt. The reaction was repeated at the same scale, and the second melt was poured into the same solution of potassium hydroxide. After the mixture was thoroughly stirred, the stirrer was turned off, and the mixture was allowed to settle.

### Synthesis of SAHA

### Step 1—Synthesis of Suberanilic Acid

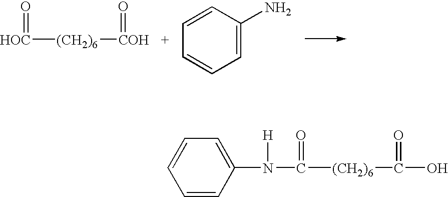

The mixture was then filtered through a pad of Celite (4,200 g). The product was filtered to remove the neutral by-product from attack by aniline on both ends of suberic acid. The filtrate contained the salt of the product, and also the salt of unreacted suberic acid. The mixture was allowed to settle because the filtration was very slow, taking several days. The filtrate was acidified using 5 L of concentrated hydrochloric acid; the mixture was stirred for one hour, and then allowed to settle overnight. The product was collected by filtration, and washed on the funnel with deionized water (4×5 L). The wet filter cake was placed in a 72 L flask with 44 L of deionized water, the mixture heated to 50° C., and the solid isolated by a hot filtration (the desired product was contaminated with suberic acid which is has a much greater solubility in hot water. Several hot triturations were done to remove suberic acid. The product was checked by NMR [D₆DMSO] to monitor the removal of suberic acid). The hot trituration was repeated with 44 L of water at 50° C. The product was again isolated by filtration, and rinsed with 4 L of hot water. It was dried over the weekend in a vacuum oven at 65° C. using a Nash pump as the vacuum source (the Nash pump is a liquid ring pump (water) and pulls a vacuum of about 29 inch of mercury. An intermittent argon purge was used to help carry off water); 4,182.8 g of suberanilic acid was obtained.

The product still contained a small amount of suberic acid; therefore the hot trituration was done portionwise at 65° C., using about 300 g of product at a time. Each portion was filtered, and rinsed thoroughly with additional hot water (a total of about 6 L). This was repeated to purify the entire batch. This completely removed suberic acid from the product. The solid product was combined in a flask and stirred with 6 L of methanol/water (1:2), and then isolated by filtration and air dried on the filter over the week end. It was placed in trays and dried in a vacuum oven at 65° C. for 45 hours using the Nash pump and an argon bleed. The final product has a weight of 3,278.4 g (32.7% yield).

### Step 2—Synthesis of Methyl Suberanilate

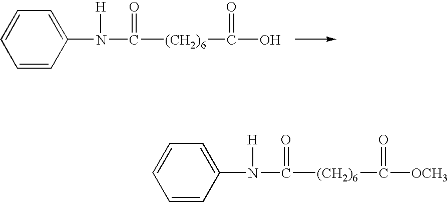

To a 50 L flask fitted with a mechanical stirrer, and condenser was placed 3,229 g of suberanilic acid from the previous step, 20 L of methanol, and 398.7 g of Dowex 50WX2-400 resin. The mixture was heated to reflux and held at reflux for 18 hours. The mixture was filtered to remove the resin beads, and the filtrate was taken to a residue on a rotary evaporator.

The residue from the rotary evaporator was transferred into a 50 L flask fitted with a condenser and mechanical stirrer. To the flask was added 6 L of methanol, and the mixture heated to give a solution. Then 2 L of deionized water was added, and the heat turned off. The stirred mixture was allowed to cool, and then the flask was placed in an ice bath, and the mixture cooled. The solid product was isolated by filtration, and the filter cake was rinsed with 4 L of cold methanol/water (1:1). The product was dried at 45° C. in a vacuum oven using a Nash pump for a total of 64 hours to give 2,850.2 g (84% yield) of methyl suberanilate.

### Step 3—Synthesis of Crude SAHA

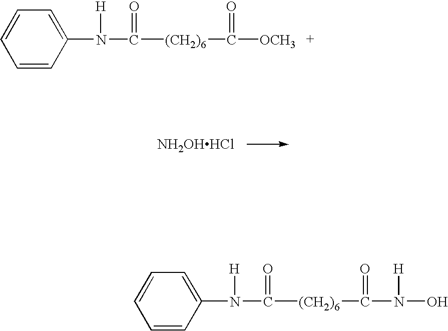

To a 50 L flask with a mechanical stirrer, thermocouple, and inlet for inert atmosphere was added 1,451.9 g of hydroxylamine hydrochloride, 19 L of anhydrous methanol, and a 3.93 L of a 30% sodium methoxide solution in methanol. The flask was then charged with 2,748.0 g of methyl suberanilate, followed by 1.9 L of a 30% sodium methoxide solution in methanol. The mixture was allowed to stir for 16 hr and 10 minutes. Approximately one half of the reaction mixture was transferred from the reaction flask (flask 1) to a 50 L flask (flask 2) fitted with a mechanical stirrer. Then 27 L of deionized water was added to flask 1 and the mixture was stirrer for 10 minutes. The pH was taken using a pH meter; the pH was 11.56. The pH of the mixture was adjusted to 12.02 by the addition of 100 ml of the 30% sodium methoxide solution in methanol; this gave a clear solution (the reaction mixture at this time contained a small amount of solid. The pH was adjusted to give a clear solution from which the precipitation the product would be precipitated). The reaction mixture in flask 2 was diluted in the same manner; 27 L of deionized water was added, and the pH adjusted by the addition of 100 ml of a 30% sodium methoxide solution to the mixture, to give a pH of 12.01 (clear solution).

The reaction mixture in each flask was acidified by the addition of glacial acetic acid to precipitate the product. Flask 1 had a final pH of 8.98, and Flask 2 had a final pH of 8.70. The product from both flasks was isolated by filtration using a Buchner funnel and filter cloth. The filter cake was washed with 15 L of deionized water, and the funnel was covered and the product was partially dried on the funnel under vacuum for 15.5 hr. The product was removed and placed into five glass trays. The trays were placed in a vacuum oven and the product was dried to constant weight. The first drying period was for 22 hours at 60° C. using a Nash pump as the vacuum source with an argon bleed. The trays were removed from the vacuum oven and weighed. The trays were returned to the oven and the product dried for an additional 4 hr and 10 minutes using an oil pump as the vacuum source and with no argon bleed. The material was packaged in double 4-mill polyethylene bags, and placed in a plastic outer container. The final weight after sampling was 2633.4 g (95.6%).

### Step 4—Recrystallization of Crude SAHA

The crude SAHA was recrystallized from methanol/water. A 50 L flask with a mechanical stirrer, thermocouple, condenser, and inlet for inert atmosphere was charged with the crude SAHA to be crystallized (2,525.7 g), followed by 2,625 ml of deionized water and 15,755 ml of methanol. The material was heated to reflux to give a solution. Then 5,250 ml of deionized water was added to the reaction mixture. The heat was turned off, and the mixture was allowed to cool. When the mixture had cooled sufficiently so that the flask could be safely handled (28° C.), the flask was removed from the heating mantle, and placed in a tub for use as a cooling bath. Ice/water was added to the tub to cool the mixture to −5° C. The mixture was held below that temperature for 2 hours. The product was isolated by filtration, and the filter cake washed with 1.5 L of cold methanol/water (2:1). The funnel was covered, and the product was partially dried under vacuum for 1.75 hr. The product was removed from the funnel and placed in 6 glass trays. The trays were placed in a vacuum oven, and the product was dried for 64.75 hr at 60° C. using a Nash pump as the vacuum source, and using an argon bleed. The trays were removed for weighing, and then returned to the oven and dried for an additional 4 hours at 60° C. to give a constant weight. The vacuum source for the second drying period was an oil pump, and no argon bleed was used. The material was packaged in double 4-mill polyethylene bags, and placed in a plastic outer container. The final weight after sampling was 2,540.9 g (92.5%).

In other experiments, crude SAHA was crystallized using the following conditions:

TABLE 1
SAHA Crystallization Conditions

| Solvent | Water | Agitation | Time (hr) |
|---|---|---|---|
| Methanol | — | Off | 2 |
| Methanol | — | On | 72 |
| Ethanol | — | On | 72 |
| Isopropanol | — | Off | 72 |
| Ethanol | 15% | On | 2 |
| Methanol | 15% | Off | 72 |
| Ethanol | 15% | Off | 72 |
| Ethanol | 15% | On | 72 |
| Methanol | 15% | On | 72 |

All these reaction conditions produced SAHA Polymorph I.

### Example 2

### Generation of Wet-Milled Small Particles in 1:1 Ethanol/Water

The SAHA Polymorph I crystals were suspended in 1:1 (by volume) EtOH/water solvent mixture at a slurry concentration ranging from 50 mg/gram to 150 mg/gram (crystal/solvent mixture). The slurry was wet milled with IKA-Works Rotor-Stator high shear homogenizer model T50 with superfine blades at 20-30 m/s, until the mean particle size of SAHA was less than 50 μm and 95% less than 100 μm, while maintaining the temperature at room temperature. The wet-milled slurry was filtered and washed with the 1:1 EtOH/water solvent mixture at room temperature. The wet cake was then dried at 40° C. The final mean particle size of the wet-milled material was less than 50 μm as measured by the Microtrac method below.

Particle size was analyzed using an SRA-150 laser diffraction particle size analyzer, manufactured by Microtrac Inc. The analyzer was equipped with an ASVR (Automatic Small Volume Recirculator). 0.25 wt % lecithin in ISOPAR G was used as the dispersing fluid. Three runs were recorded for each sample and an average distribution was calculated. Particle size distribution (PSD) was analyzed as a volume distribution. The mean particle size and 95%<values based on volume were reported.

### Example 2A

### Large Scale Generation of Wet-Milled Small Particles in 1:1 Ethanol/Water

56.4 kg SAHA Polymorph I crystals were charged to 610 kg (10.8 kg solvent per kg SAHA) of a 50% vol/vol solution of 200 proof punctilious ethanol and water (50/50 EtOH/Water) at 20-25° C. The slurry (˜700 L) was recirculated through an IKA Works wet-mill set with super-fine generators until reaching a steady-state particle size distribution. The conditions were: DR3-6, 23 m/s rotor tip speed, 30-35 Lpm, 3 gen, ˜96 turnovers (a turnover is one batch volume passed through one gen), ˜12 hrs.

$$Approx . Mill Time ( hr ) = \frac{96 \times Batch Volume ( L )}{\begin{matrix}Natural Draft of Mill ( Lpm ) \times \\ \# of Generators \times 60\end{matrix}}$$

The wet cake was filtered, washed 2× with water (total 6 kg/kg, ˜340 kg) and vacuum dried at 40-45° C. The dry cake was then sieved (595 μm screen) and packed as Fine API.

### Example 3

### Growth of Large Crystals of Mean Particle Size 150 μm in 1:1 Ethanol/Water

25 grams of SAHA Polymorph I crystals and 388 grams of 1:1 Ethanol/water solvent mixture were charged into a 500 ml jacketed resin kettle with a glass agitator. The slurry was wet milled to a particle size less than 50 μm at room temperature following the steps of Example 2. The wet-milled slurry was heated to 65° C. to dissolve ˜85% of the solid. The heated slurry was aged at 65° C. for 1-3 hours to establish a ˜15% seed bed. The slurry was mixed in the resin kettle under 20 psig pressure, and at an agitator speed range of 400-700 rpm.

The batch was then cooled slowly to 5° C.: 65 to 55° C. in 10 hours, 55 to 45° C. in 10 hours, 45 to 5° C. in 8 hours. The cooled batch was aged at 5° C. for one hour to reach a target supernatant concentration of less than 5 mg/g, in particular, 3 mg/g. The batch slurry was filtered and washed with 1:1 EtOH/water solvent mixture at 5° C. The wet cake was dried at 40° C. under vacuum. The dry cake had a final particle size of ˜150 μm with 95% particle size <300 μm according to the Microtrac method.

### Example 4

### Growth of Large Crystals with Mean Particle Size of 140 μm in 1:1 Ethanol/Water

7.5 grams of SAHA Polymorph I crystals and 70.7 grams of 1:1 EtOH/water solvent mixture were charged into a seed preparation vessel (500-ml jacketed resin kettle). The seed slurry was wet milled to a particle size less than 50 μm at room temperature following the steps of Example 2 above. The seed slurry was heated to 63-67° C. and aged over 30 minutes to 2 hours.

In a separate crystallizer (1-liter jacketed resin kettle), 17.5 grams of SAHA Polymorph I crystals and 317.3 grams of 1:1 EtOH/water solvent mixture were charged. The crystallizer was heated to 67-70° C. to dissolve all solid SAHA crystals first, and then was cooled to 60-65° C. to keep a slightly supersaturated solution.

The seed slurry from the seed preparation vessel was transferred to the crystallizer. The slurry was mixed in the resin kettle under 20 psig pressure, and at an agitator speed range similar to that in Example 3. The batch slurry was cooled slowly to 5° C. according to the cooling profile in Example 3. The batch slurry was filtered and washed with 1:1 EtOH/water solvent mixture at 5° C. The wet cake was dried at 40° C. under vacuum. The dry cake had a final particle size of about 140 μm with 95% particle size <280 μm.

### Example 4A

### Large Scale Growth of Large Crystals in 1:1 Ethanol/Water

21.9 kg of the Fine API dry cake from Example 2A (30% of total) and 201 kg of 50/50 EtOH/Water solution (2.75 kg solvent/kg total SAHA) was charged to Vessel #1—the Seed Preparation Tank. 51.1 kg of SAHA Polymorph I crystals (70% of total) and 932 kg 50/50 EtOH/Water (12.77 kg solvent/kg total SAHA) was charged to Vessel #2—the Crystallizer. The Crystallizer was pressurized to 20-25 psig and the contents heated to 67-70° C. while maintaining the pressure to fully dissolve the crystalline SAHA. The contents were then cooled to 61-63° C. to supersaturate the solution. During the aging process in the Crystallizer, the Seed Prep Tank was pressurized to 20-25 psig, the seed slurry was heated to 64° C. (range: 62-66° C.), aged for 30 minutes while maintaining the pressure to dissolve ˜2 of the seed solids, and then cooled to 61-63° C.

The hot seed slurry was rapidly transferred from the Seed Prep Tank to the Crystallizer (no flush) while maintaining both vessel temperatures. The nitrogen pressure in the Crystallizer was re-established to 20-25 psig and the batch was aged for 2 hours at 61-63° C. The batch was cooled to 5° C. in three linear steps over 26 hours: (1) from 62° C. to 55° C. over 10 hours; (2) from 55° C. to 45° C. over 6 hours; and (3) from 45° C. to 5° C. over 10 hours. The batch was aged for 1 hr and then the wet cake was filtered and washed 2× with water (total 6 kg/kg, ˜440 kg), and vacuum dried at 40-45° C. The dry cake from this recrystallization process is packed-out as the Coarse API. Coarse API and Fine API were blended at a 70/30 ratio.

### Example 5

### Generation of Wet-Milled Small Particles Batch 288

SAHA Polymorph I crystals were suspended in ethanolic aqueous solution (100% ethanol to 50% ethanol in water by volume) at a slurry concentration ranging from 50 mg/gram to 150 mg/gram (crystal/solvent mixture). The slurry was wet milled with IKA-Works Rotor-Stator high shear homogenizer model T50 with superfine blades at 20-35 m/s, until the mean particle size of SAHA was less than 50 μm and 95% less than 100 μm, while maintaining the temperature at room temperature. The wet-milled slurry was filtered and washed with EtOH/water solvent mixture at room temperature. The wet cake was then dried at 40° C. The final mean particle size of the wet-milled material was less than 50 μm as measured by the Microtrac method as described before.

### Example 6

### Growth of Large Crystals Batch 283

24 grams of SAHA Polymorph I crystals and 205 ml of 9:1 Ethanol/water solvent mixture were charged into a 500 ml jacketed resin kettle with a glass agitator. The slurry was wet milled to a particle size less than 50 μm at room temperature following the steps of Example 1. The wet-milled slurry was heated to 65° C. to dissolve ˜85% of the solid. The heated slurry was aged at 64-65° C. for 1-3 hours to establish a ˜15% seed bed. The slurry was mixed at an agitator speed range of 100-300 rpm.

The batch was then cooled to 20° C. with one heat-cool cycle: 65° C. to 55° C. in 2 hours, 55° C. for 1 hour, 55° C. to 65° C. over ˜30 minutes, age at 65° C. for 1 hour, 65° C. to 40° C. in 5 hours, 40° C. to 30° C. in 4 hours, 30° C. to 20° C. over 6 hours. The cooled batch was aged at 20° C. for one hour. The batch slurry was filtered and washed with 9:1 EtOH/water solvent mixture at 20° C. The wet cake was dried at 40° C. under vacuum. The dry cake had a final particle size of ˜150 μm with 95% particle size <300 μm per Microtrac method.

30% of the batch 288 crystals and 70% of the batch 283 crystals were blended to produce capsules containing about 100 mg of suberoylanilide hydroxamic acid; about 44.3 mg of microcrystalline cellulose; about 4.5 mg of croscarmellose sodium; and about 1.2 mg of magnesium stearate.

### Example 7

### Assays for Viability of Multiple Myeloma Cell Lines Treated with SAHA and Bortezomib

For these experiments, the effect of the vorinostat/bortezomib combination was evaluated across 4 cell lines. Cells were left untreated (control) or treated with vorinostat, bortezomib or the combination at the concentrations indicated. In the case of the combination, the cells were subjected to bortezomib treatment for 6 h, at which point vorinostat was added to the incubation media. Viability of the cells was assessed by standard AlamarBlue assay 48 h post-initiation of treatment.

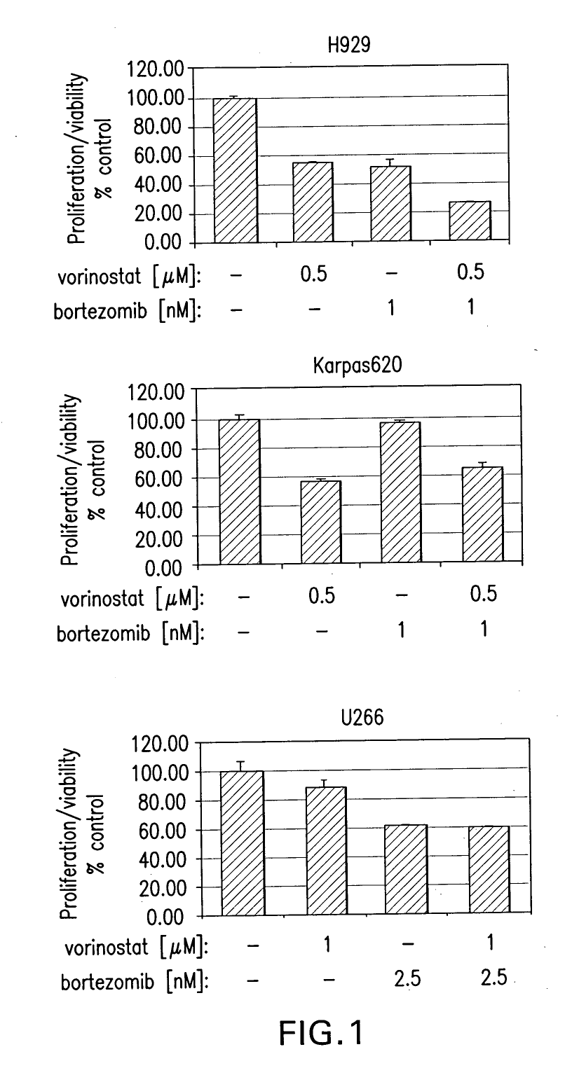

The effects of simultaneous drug treatment as well as various staggered schedules in which cells where preincubated with one of the agents followed by addition of the second agent were tested. Further, complete dose-response curves for the single agents and combination were tested. For the purpose of illustration, a few defined pairs of concentrations are shown in FIG. 1. The results from these experiments indicate that in H929 cells a 6 h period with bortezomib followed by addition of vorinostat results in a “subadditive” (less than additive, but superior to either agent alone) effect on cell proliferation/viability upon 48 h from initiation of treatment. Similar results were observed in SKMM2 cells. In the case of Karpas620 cells, bortezomib pretreatment did not have an added benefit with respect to vorinostat single agent. In contrast, in the U266 cells the combination did not show superior activity compared to bortezomib alone. Thus, the data suggest that in cell lines where both agents show efficacy when individually administered, the combination of suboptimal concentrations has an enhanced anti-proliferative activity. On the other hand, in cells that show very poor response to one of the compounds the overall response is driven by the most effective compound.

### Example 8

### Phase I Clinical Trial of Oral SAHA in Combination with Bortezomib in Patients with Advanced Multiple Myeloma

This study is used to determine the maximum tolerated dose (MTD) for the combination of oral SAHA (Vorinostat) and standard doses of Bortezomib in patients with advanced multiple myeloma. The study is designed to assess the pharmacokinetics of SAHA alone and when administered in combination with Bortezomib. The study is used to assess the safety and tolerability of the combination regimen of SAHA and Bortezomib. The study is also used estimate response rate, time to response, response duration, progression-free survival, and time to progression for SAHA and Bortezomib when used in combination. In this study, administration of SAHA in combination with Bortezomib to patients with advanced multiple myeloma is tested for sufficient safety and tolerance to permit further study.

Study Design and Duration: This is a multicenter, open label, escalating dose, Phase I study of SAHA in combination with intravenous Bortezomib injection in patients with advanced multiple myeloma who would be eligible for Bortezomib therapy. In this non-randomized trial, patients on Dose Levels 1 and 2 are treated with SAHA for 7 days, followed by a 14 day rest period, for a 21 day treatment cycle for up to 8 cycles. Bortezomib is administered as an intravenous (IV) bolus on Days 1 and 4 for Dose Level 1 and days 1, 4, 8, and 11 for Dose Level 2. Patients on subsequent dose levels are treated with SAHA for 14 days, followed by a 7 day rest period, for a 21 day treatment cycle for up to 8 cycles. Bortezomib is administered on Days 1, 4, 8, and 11. Patients who are enrolled on the previous version of the protocol, are treated only at the initial dose level specified in that version. Patients who experience progressive disease or intolerable toxicity are discontinued. Patients who do not have disease progression and who continue to meet the eligibility criteria after the first 8 cycles, are offered continued treatment with SAHA at the same dose and schedule on a continuation protocol.

Patient Sample: Up to 40 adult patients with advanced multiple myeloma (relapsed or refractory disease) are enrolled. A minimum of 3 and a maximum of 6 patients are enrolled at each dose level to establish the maximum tolerated dose (MTD) of the combination therapy. Once the MTD is established, an additional 6 patients are enrolled at recommended Phase II dose, to study the pharmacokinetics of the regimen. Eligible patients must be ≧18 years; have ECOG Performance Status of 0-2; adequate hematologic, hepatic, and renal function; ability to swallow capsules; ≧3 weeks from prior chemotherapy, radiation therapy, major surgery, or other investigational anticancer therapy; and have recovered from prior toxicities.

Dosage/Dosage Form, Route, and Dose Regimen: One treatment cycle is 3 weeks or 21 days. Patients on Dose Levels 1 and 2 are treated with SAHA 400 mg P.O. daily (q.d.) for 7 days, followed by a 14 day rest period, for a 21 day treatment cycle. Bortezomib 0.7 mg/m² is administered as an intravenous (IV) bolus on Days 1 and 4 for Dose Level 1 and days 1, 4, 8, and 11 for Dose Level 2. Patients on subsequent dose levels are treated with SAHA 400 mg P.O. daily (q.d.) for 14 days, followed by a 7 day rest period, for a 21 day treatment cycle. Bortezomib 0.7-1.3 mg/m² IV bolus is administered on Days 1, 4, 8, and 11 (see Table 14). Patients who are enrolled on the previous version of the protocol, continue treatment only at the initial dose level of SAHA 200 mg P.O. b.i.d. for 14 days, followed by a 7 day rest and Bortezomib 0.7 mg/m² IV bolus on days 4, 8, 11, and 15. On days where SAHA and Bortezomib are administered concurrently, the SAHA dose is given prior to the Bortezomib administration. Although the current FDA approved Bortezomib dose for relapsed myeloma patients is 1.3 mg/m², for purposes of this initial investigation of SAHA in combination with Bortezomib, subjects enrolled at the first dose level receive Bortezomib at 0.7 mg/m². If the combination therapy is found to be safe, then dose escalation proceeds.

The starting dose level of SAHA (Dose Level 1) is 400 mg P.O. q.d. for 7 days followed by 14 days rest, for a complete treatment cycle of 21 days. Other potential dose levels are defined in the Table below.

TABLE 14
Once Daily (q.d.) Dosing Schedule for SAHA in Combination with Bortezomib

| Dose | | | Dose Modification | | |
|---|---|---|---|---|---|
| Level | SAHA Dose (mg) | Bortezomib Dose (mg/m²) | SAHA Dose (mg) | | Bortezomib Dose (mg/m²) |
| 1 | 400 × 7 days | 0.7 on Days 1, 4 | N/A | | N/A |
| 2 | 400 × 7 days | 0.7 on Days 1, 4, 8, 11 | 400 × 7 days | | 0.7 on Days 1, 4 |
| 3 | 400 × 14 days | 0.7 on Days 1, 4, 8, 11 | A. | 400 × 10 days | 0.7 on Days 1, 4, 8, 11 |
| | | | then | | |
| | | | B. | 400 × 7 days | 0.7 on Days 1, 4, 8, 11 |
| 4 | 400 × 14 days | 0.9 on Days 1, 4, 8, 11 | A. | 400 × 14 days | 0.7 on Days 1, 4, 8, 11 |
| | | | then | | |
| | | | B. | 400 × 10 days | 0.7 on Days 1, 4, 8, 11 |
| 5 | 400 × 14 days | 1.1 on Days 1, 4, 8, 11 | A. | 400 × 14 days | 0.9 on Days 1, 4, 8, 11 |
| | | | then | | |
| | | | B. | 400 × 10 days | 0.9 on Days 1, 4, 8, 11 |
| 6 | 400 × 14 days | 1.3 on Days 1, 4, 8, 11 | A. | 400 × 14 days | 1.1 on Days 1, 4, 8, 11 |
| | | | then | | |
| | | | B. | 400 × 10 days | 1.1 on Days 1, 4, 8, 11 |

Note:
Treatment cycle is defined as 21 days or 3 weeks. Therefore, 7 consecutive days of SAHA is followed by 14 days rest; 14 consecutive days of SAHA is followed by 7 days rest; and 10 consecutive days of SAHA is followed by 11 days rest.

Barring dose-limiting-toxicities (DLTs), the dose is escalated from Dose Level 1 up to Dose Level 6. Dosing in this study does not exceed Dose Level 6.

The given procedure is followed. If Dose Level 1 is greater than the MTD, then the study is terminated. If Dose Level 1 is well tolerated, then dose escalation proceeds to Dose Level 2. If Dose Level 2 is greater than the MTD, then Dose Level 1 is considered the MTD and expanded to a total enrollment of 6 patients per MTD cohort. If Dose Level 2 is well tolerated, then dose escalation proceeds to Dose Level 3. If Dose Level 3 is greater than the MTD, then Dose Level 2 is considered the MTD and expanded to a total enrollment of 6 patients per MTD cohort. If Dose Level 3 is well tolerated, then dose escalation proceeds to Dose Level 4. If Dose Level 4 is greater than the MTD, then Dose Level 3 is considered the MTD and expanded to a total enrollment of 6 patients per MTD cohort. If Dose Level 4 is well tolerated, then dose escalation proceeds to Dose Level 5. If Dose Level 5 is greater than the MTD, then Dose Level 4 is considered the MTD and expanded to a total enrollment of 6 patients per MTD cohort. If Dose Level 5 is well tolerated, then dose escalation proceeds to Dose Level 6. If Dose Level 6 is greater than the MTD, then Dose Level 5 is considered the MTD and expanded to a total enrollment of 6 patients per MTD cohort. If Dose Level 6 is well tolerated, then it is considered the MTD and expanded to a total enrollment of 6 patients per MTD cohort. The investigator consults with the medical monitor prior to making dose adjustments with SAHA or Bortezomib.

Once the MTD has been established, the recommended Phase II dose is studied in 6 additional patients. The recommended Phase II dose is at MTD or below as determined following review of all safety, pharmacodynamics, and efficacy data obtained over repeated cycles in this study. In addition, review of safety across repeated cycles can influence decisions on dose escalation.

Efficacy Measurements: Patients' clinical status (by antitumor activity) for this combination is documented using the European Group for Blood and Marrow Transplantation (EBMT) criteria (Blade, J., et al. (1998) British J. Haematol. 102 (5), 1115-1123). The study is used to estimate response rate, time to response, response duration, and time to progression for SAHA and Bortezomib when used in combination. The investigator monitors disease progression/response every 2 cycles or more frequently, if appropriate and reports accordingly.

Safety Measurements: Safety parameters consisting of assessment of vital signs, physical examination, ECOG performance status, adverse events, serious adverse events, laboratory safety tests and electrocardiograms are obtained or assessed prior to drug administration and at designated intervals throughout the study.

Treatment Plan: At dose level, the appropriate number of 100-mg capsules of SAHA is to be administered orally in repeated 21-day cycles consisting of 7-14 days dosing followed by a 7-14-day rest period, during which no SAHA is administered. During the dosing period, the capsules are taken with food (within 30 minutes following a meal), whenever possible. The total dose consumed at any one time is not to exceed the assigned dose, and missed doses are not made up. Sufficient drug for 7 days of treatment is dispensed for patients on Dose Levels 1 and 2 at the beginning of each 21-day cycle. Subsequent dose levels have a 14 day supply of drug dispensed at the beginning of each 21-day cycle. Any unused drug is returned to the site at the completion of the dosing period of the cycle. A capsule count is performed at the completion of each cycle and end of study visit to monitor compliance.

Bortezomib injection is administered as IV bolus on days 1 and 4 of the initial dose level and on Days 1, 4, 8, and 11 of each subsequent dose levels. On days where SAHA and Bortezomib are administered concurrently, the SAHA dose is given prior to the Bortezomib administration. Subjects enrolled at the first dose level receive Bortezomib at 0.7 mg/m². If the combination therapy is found to be safe, then dose escalation proceeds. Other Bortezomib doses in this study are 0.9, 1.1, and 1.3 mg/m². Prior to Bortezomib administration of each cycle on Days 1 and 4 on Dose Level 1 and Days 1, 4, 8, and 11 on Dose Levels 2-6, absolute neutrophil count (ANC) is expected at ≧1,000/μL and platelet count ≧50,000/μL.

Clinical Laboratory Tests: Different clinical laboratory tests are performed at screening and Days 1, 8, 11, and 15 of all cycles. Laboratory tests include the measurements for hematology, chemistry, coagulation, and urinalysis. Also included are myeloma disease measurements, in particular: serum protein electrophoresis, quantitative immunoglobulins, serum immunofixation, 24 hr urine protein electrophoresis and urine immunofixation. Other serum tests are also included: β-hCG (only in women of child bearing potential), β2 microglobulin, and C-reactive protein. Any treatment-emergent clinically significant clinical laboratory abnormality is reported and followed.

Pharmacokinetic (PK) Samples in Patients Enrolled at Recommended Phase II Dose Level: Once MTD is established, 6 new patients are enrolled to study the recommended Phase II dose in this patient population. The recommended Phase II dose is at MTD or below as determined following review of all safety, pharmacodynamics, and efficacy data obtained over repeated cycles in this study. In addition, review of safety across repeated cycles can be used to influence decisions on dose escalation. Pharmacokinetic measurements are studied only in this group of patients. On Cycle 1 Day 3, SAHA PK samples are collected before dosing, 15 minutes postdose and 30 minutes postdose. PK samples continue to be collected at 1, 2, 3, 5, 8, 10, and 12 hours postdose. On Cycle 1 Day 11, the patient receives their morning dose of SAHA in clinic followed by an immediate administration of IV bolus of Bortezomib. The SAHA PK samples are collected before dosing, 15 minutes postdose and 30 minutes postdose. SAHA PK samples continue to be collected at 1, 2, 3, 5, 8, 10, and 12 hours postdose. In addition, Bortezomib PK samples are collected before dosing and at 5, 10, 15, and 30 minutes postdose. Bortezomib PK samples continue to be collected at 1, 2, 3, 5, 8, 10, 12, and 24 hours postdose. The Bortezomib PK samples are for archive purposes. PK parameters include area under the concentration-time curve (AUC), maximum plasma or serum concentration (Cₘₐₓ), time to maximum plasma or serum concentration (Tₘₐₓ), and apparent half-life (t_{1/2}). The PK parameters (AUC₀₋₁₂, Cₘₐₓ, and Tₘₐₓ) of SAHA and PK parameters (AUC_{0-inf}, Cₘₐₓ, Tₘₐₓ, and apparent t_{1/2}) of Bortezomib are provided upon analysis of the PK samples.

Specific Doses, Dose Escalation, and Modification for SAHA and Bortezomib: The starting dose level of SAHA (Dose Level 1) is 400 mg P.O q.d. for 7 days followed by 14 days of rest, for a complete treatment cycle of 21 days. Other potential dose levels and dose modifications are defined in the Table, above. If the dosage for SAHA at 400 mg P.O. q.d.×14 days and Bortezomib at 0.7 mg/m² is not tolerated, the SAHA dosage is de-escalated to 400 mg P.O q.d.×10 days. If the SAHA dosage at 400 mg P.O q.d.×10 days is not tolerated, the second de-escalation for SAHA is set to 400 mg P.O q.d.×7 days. For patients on dose levels receiving SAHA 400 mg P.O. q.d.×14 days with Bortezomib escalation to 0.9, 1.1, and 1.3 mg/m², the first de-escalation sets back one dose level of Bortezomib. The second de-escalation is set to SAHA 400 mg P.O. q.d.×10 days.

### Example 9

### Phase I/II Clinical Trial of Oral SAHA in Combination with Bortezomib in Patients with Advanced Multiple Myeloma

This study was used to determine the maximum tolerated dose (MTD) for the combination of oral vorinostat and standard doses of Bortezomib in patients with advanced multiple myeloma. Furthermore, the study was used to assess the safety and tolerability of the combination regimen of Vorinostat and Bortezomib, to estimate response rate, time to response, and response and duration and time to progression for Vorinostat and Bortezomib when used in combination.

This was a multicenter, open label, escalating dose, Phase I study of vorinostat in combination with intravenous Bortezomib injection in patients with advanced multiple myeloma who would be eligible for Bortezomib therapy. In this non-randomized trial, patients were treated with vorinostat for 14 days, followed by a 7-day rest period, for a 21-day treatment cycle for up to 8 cycles. Patients on Dose Levels 1 and 2 were administered Bortezomib as an intravenous (IV) bolus on Days 4, 8, 11 and 15. Patients on subsequent dose levels were administered Bortezomib as an intravenous (IV) bolus on Days 1, 4, 8, 11. Patients who completed at least 1 cycle of treatment with Vorinostat in combination with Bortezomib and then experienced progressive disease may be treated with dexamethasone 20 mg p.o. daily on Days 1-4, and 9-12 of each cycle along with Vorinostat and Bortezomib as scheduled.

If further progressive disease was experienced after two treatment cycles of Vorinostat, Bortezomib and dexamethasone, the patient was going to be discontinued permanently. Patients who experienced intolerable toxicity were discontinued. Patients who did not have disease progression and who continued to meet the eligibility criteria after the first 8 cycles, were offered continued treatment with vorinostat at the same dose and schedule.

These studies enrolled up to 40 adult patients with advanced multiple myeloma (relapsed or refractory disease). A minimum of 3 and a maximum of 6 patients were enrolled at each dose level to establish the maximum tolerated dose (MTD) of the combination therapy. Once the MTD was established, an additional 6 patients were enrolled at recommended Phase II dose, to study the pharmacokinetics of the regimen. Eligible patients were ≧18 years;

One treatment cycle was 3 weeks or 21 days. The Vorinostat capsules were given orally (p.o.) b.i.d. for 14 consecutive days (Day 1 through Day 14). Bortezomib injection were administered as an intravenous (IV) bolus twice weekly for two weeks in each cycle. On days where Vorinostat and Bortezomib were administered concurrently, the vorinostat dose was given prior to the Bortezomib administration. Although the current FDA approved Bortezomib dose for relapsed myeloma patients was 1.3 mg/m², for safety purposes in this initial investigation of vorinostat in combination with Bortezomib, subjects enrolled at the first dose level receive Bortezomib at 0.7 mg/m². If the combination therapy was found to be safe, then dose escalation would proceed. Other Bortezomib doses in this study were 0.9, 1.1 and 1.3 mg/m². Patients on Dose Levels 1 and 2 were treated with 200 mg b.i.d. of Vorinostat p.o. for 14 days, followed by a 7-day rest period, in a 21-day treatment cycle. Treatment with Vorinostat could be for up to 8 cycles. Bortezomib was administered as an intravenous (IV) bolus on Days 4, 8, 11, and 15. Patients on subsequent dose levels were treated with Vorinostat p.o. at a dose of 400 mg q.d. for 14 days, followed by a 7-day rest period, in a 21-day treatment cycle. Treatment with Vorinostat could be for up to 8 cycles. Bortezomib was administered on Days 1, 4, 8, and 11. Please refer to Table 1 below.

TABLE 1

| Dose | | | Vorinostat Total |
|---|---|---|---|
| Level | Bortezomib Dose | Vorinostat Dose | Daily Dose (mg) |
| 1 | 0.7 mg/m² days 4, 8, 11, 15 | 200 mg b.i.d. × 14 days | 400 |
| 2 | 0.9 mg/m² days 4, 8, 11, 15 | 200 mg b.i.d. × 14 days | 400 |
| 3 | 0.9 mg/m² days 1, 4, 8, 11 | 400 mg q.d. × 14 days | 400 |
| 4 | 1.1 mg/m² days 1, 4, 8, 11 | 400 mg q.d. × 14 days | 400 |
| 5 | 1.3 mg/m² days 1, 4, 8, 11 | 400 mg q.d. × 14 days | 400 |

Barring DLTs, the dose was escalated from Dose Level 1 up to Dose Level 5. Dosing in this study did not exceed Dose Level 5.

The procedure below was followed:

If Dose Level 1 was greater than the MTD, then the study was terminated.

If Dose Level 1 was well tolerated, then dose escalation proceeded to Dose Level 2.
If Dose Level 2 was greater than the MTD, then Dose Level I was considered the MTD and expanded to a total enrollment of 6 patients per MTD cohort.

If Dose Level 2 was well tolerated, then dose escalation proceeded to Dose Level 3.
If Dose Level 3 was greater than the MTD, then Dose Level 2 was considered the MTD and expanded to a total enrollment of 6 patients per MTD cohort.

If Dose Level 3 was well tolerated, then dose escalation proceeded to Dose Level 4.
If Dose Level 4 was greater than the MTD, then Dose Level 3 was considered the MTD and expanded to a total enrollment of 6 patients per MTD cohort.

If Dose Level 4 was well tolerated, then dose escalation proceeded to Dose Level 5.
If Dose Level 5 was greater than the MTD, then Dose Level 4 would be considered the MTD and expanded to a total enrollment of 6 patients per MTD cohort.

If Dose Level 5 was well tolerated, then it would be considered the MTD and expanded to a total enrollment of 6 patients per MTD cohort.

Once the MTD had been established, the recommended Phase II dose was studied in 6 additional patients. The recommended Phase II dose was at MTD or below as determined following review of all, safety, pharmacodynamics and efficacy data obtained over repeated cycles in this study.

While this invention has been particularly shown and described with references to particular embodiments thereof, it will be understood by those skilled in the art that various changes in form and details may be made therein without departing from the meaning of the invention described. The scope of the invention encompasses the claims that follow.

1. A method of treating multiple myeloma in a patient comprising administering to the patient: i) SAHA (suberoylanilide hydroxamic acid), represented by the structure: 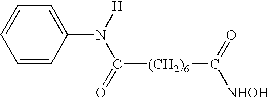
or a pharmaceutically acceptable salt or hydrate thereof by oral administration once daily at a dose of 400 mg on Days 1-14 out of 21 days; and ii) (1R)-3-methyl-1-[[(2S)-1-oxo-3-phenyl-2-[(pyrazinylcarbonyl)amino]propyl]amino]butyl]boronic acid (Bortezomib) or a pharmaceutically acceptable salt or hydrate thereof by intravenous bolus administration at a dose of 0.9 mg/m² on Days 1, 4, 8 and 11 out of 21 days.

2. (canceled)

3. (canceled)

4. (canceled)

5. (canceled)

6. (canceled)

7. (canceled)

8. The method of claim 13 wherein the administration of SAHA or pharmaceutically acceptable salt or hydrate thereof is repeated for up to eight treatment periods of 21 days.

9. (canceled)

10. (canceled)

11. (canceled)

12. A method of treating multiple myeloma in a patient comprising administering to the patient: i) SAHA (suberoylanilide hydroxamic acid), represented by the structure: 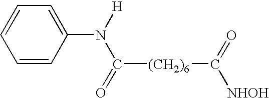
or a pharmaceutically acceptable salt or hydrate thereof by oral administration once daily at a dose of 400 mg on Days 1-14 out of 21 days; and ii) (1R)-3-methyl-1-[[(2S)-1-oxo-3-phenyl-2-[(pyrazinylcarbonyl)amino]propyl]amino]butyl]boronic acid (Bortezomib) or a pharmaceutically acceptable salt or hydrate thereof by intravenous bolus administration at a dose of 1.1 mg/m² on Days 1, 4, 8, and 11 out of 21 days.

13. A method of treating multiple myeloma in a patient comprising administering to the patient: i) SAHA (suberoylanilide hydroxamic acid), represented by the structure: 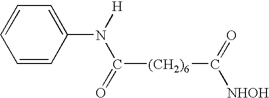
or a pharmaceutically acceptable salt or hydrate thereof by oral administration once daily at a dose of 400 mg on Days 1-14 out of 21 days; and ii) (1R)-3-methyl-1-[[(2S)-1-oxo-3-phenyl-2-[(pyrazinylcarbonyl)amino]propyl]amino]butyl]boronic acid (Bortezomib) or a pharmaceutically acceptable salt or hydrate thereof by intravenous bolus administration at a dose of 1.3 mg/m² on Days 1, 4, 8, and 11 out of 21 days.

14. (canceled)

15. (canceled)

16. (canceled)

17. (canceled)

18. (canceled)

19. The method of claim 1, wherein SAHA and Bortezomib are administered.

20. The method of claim 1 further comprising orally administering dexamethasone or a pharmaceutically acceptable salt or hydrate thereof wherein the dexamethasone or pharmaceutically acceptable salt or hydrate thereof is administered once daily at a dose of 20 mg on Days 1-4 and 9-12 for at least one treatment period of 21 days.

21. (canceled)

22. (canceled)

23. The method of claim 12 wherein SAHA and Bortezomib are administered.

24. The method of claim 13 wherein SAHA and Bortezomib are administered.

25. The method of claim 12 further comprising orally administering dexamethasone or a pharmaceutically acceptable salt or hydrate thereof wherein the dexamethasone or pharmaceutically acceptable salt or hydrate thereof is administered once daily at a dose of 20 mg on Days 1-4 and 9-12 for at least one treatment period of 21 days.

26. The method of claim 13 further comprising orally administering dexamethasone or a pharmaceutically acceptable salt or hydrate thereof wherein the dexamethasone or pharmaceutically acceptable salt or hydrate thereof is administered once daily at a dose of 20 mg on Days 1-4 and 9-12 for at least one treatment period of 21 days.

27. The method of claim 1, wherein the patient is refractory to Bortezomib.

28. The method of claim 12, wherein the patient is refractory to Bortezomib.

29. The method of claim 13, wherein the patient is refractory to Bortezomib.

30. The method of claim 1, wherein the SAHA dose is given prior to the Bortezomib administration.

31. The method of claim 12, wherein the SAHA dose is given prior to the Bortezomib administration.

33. The method of claim 13, wherein the SAHA dose is given prior to the Bortezomib administration.
